SEQUENCE LISTING (81 sequences)

SEQ ID NO: 1 - protein, 107 aa, Artificial
  Humanized Anti-RAGE Variable Region
DIQMTQSPSSLSASVGDRVTITCRASQDVGIYVNWFQQKPGKAPRRLIYRATNLADGVPSRFSGSRSGTDFTLTISSLQPEDFATYYCLEFDEHPLTFGGGTKVEIK

SEQ ID NO: 2 - protein, 119 aa, Artificial
  Humanized Anti-RAGE Variable Region
EVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDITTGFDYWGQGTLVTVSS

SEQ ID NO: 3 - protein, 242 aa, Artificial
  RAGE RD 10H6
DIQMTQSPSSLSASVGDGVTITCQASQDVGIYVNWFQQKPGKAPRRLIYRATNLADGVPSRFSGSRSGTDFTLTISSLQPEDFATYYCLEFDEHPLTFGGGTKVEIKNGGGSGGGGSGGGGSSEVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDITTGLDYWGQGTLVTESS

SEQ ID NO: 4 - protein, 242 aa, Artificial
  RAGE RD 8G9
DIQMTQSPSSLSASVGDRVTITCRASQDVGIYVNWFQQKPGKAPRRLIYRATNLADGVPSRFSGSRSGTDFTLTISSLQPEDFATYYCLEFDEHPLTFGGGTKVEIKDGGGSGGGGSGGGGSSEVQLVESGGGLVQPGSSLRLSCAASGFTFNNYWMTWVRQAPGKGLEWVASIDNSGDNTYYPDSVKDRFTISRDNAKNSLYLQMNSLRAEDTAVYYCARGGDITTGIDYWGQGTLVTVSS

SEQ ID NO: 5 - protein, 242 aa, Artificial
  RAGE RD 10D8
DIQMTQSPSSLSASVGDKVTITCRASQDVGFYVNWFQQKPGKAPRRLIYRATNLADGVPSRFSGSRSGTDFTLTISSLQPEDFATYYCLEFDEHPLTFGGGTKVEIKDGGGSGNGGSGGGGSSEVQLVESGGELVQPGGSLRLSCAASGFTFNNYWMTWVRQAPGKGLEWVASIDNSGDNTYYPDSVKDRFTISRDNAKNSLYLQMNSLRAEDTAVYYCARGGDITTGFDYWGQGTLVTVSS

SEQ ID NO: 6 - protein, 242 aa, Artificial
  RAGE RD 3B3
DIQMTQSPSSLSASVGDRVTITCRASQDVGFYVNWFQQKPGKAPRRLIYRATNLADGVPSRFSGSRSGTDFTLTISSLQPEDFATYYCLEFDEHPLTFGGGTKVEIKDGGGSGGGGSGGGGSSEVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDITTGIDYWGQGTLVTVSS

SEQ ID NO: 7 - protein, 242 aa, Artificial
  Rage RD 1G6
DIQMTQSPSSLSASVGDRVTITCRASQDVGFYVNWFQQKPGKAPRRLIYRATNLADGVPSRFSGSRSGTDFTLTISSLQPEDFATYYCLEFDEHPLTFGGGTKVEIKDGGGSGGGGSGGGGSSEVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDITNGFDYWGQGTLVTVSS

SEQ ID NO: 8 - protein, 242 aa, Artificial
  RAGE RD 2E6
DIQMTQSPSSLSASVGDRVTITCRASQDVGIYVNWFQQKPGKAPRRLIYRATNLADGVPSRFSGSRSGTDFTLTISSLQPEDFATYYCLEFDEHPLTFGGGTKVEIKDGGGSGGGGSGGGGNSEVQLVESGVGLVQPGGSLRLSCAASGFTFNNYWMTWVRQAPGKGLEWVASIDNSGDNTYYPDSVKDRFTISRDNAKNSLYLQMNSLRAEDTAVYYCARGGDITNGLDYWGQGTLVTVSS

SEQ ID NO: 9 - protein, 242 aa, Artificial
  RAGE RD 5A3
DIQMTQSPSSLSASVGDIVTITCRASQDVGIYVNWFQQKPGKAPRRLIYRATNLADGVPSRFSGSRSSTDFTLTISSLQPEDFATYYCLEFDEHPLTFGGGTKVEIKDGGGSGGGGSGGGGSSEVQLVESGGGLVQPGGSLRLSCAASGFTFNNYWMTWVRQAPGKGLEWVASIDNSGDNTYYPDSVKDRFTISRDNAKNSLYLKMNSLRAEDTAVYYCTRGGDITTGIDYWGQGTLVTVSS

SEQ ID NO: 10 - protein, 242 aa, Artificial
  RAGE RD 10G10
DIQMTQSPSSLSASVGDRVTITCRASQDVGIYVNWFQQKPGKAPRRLIYRATNLADGVPSRFSGSRSGTDFTLTISSLQPEDFATYYCLEFDEHPLTFGGGTRVEIKDGGGSGGGGSGGGGSSEVQLVESGGGLVQPGGSLRLSCAASGFTFNNYWMSWVRQAPGKGLEWVASIDNSGENTYYPDSVKDRFTISRDNAKNSLYLQMNSLRAEDTAVYYCARGGDITTGFDYWGQGTLVTVSS

SEQ ID NO: 11 - protein, 242 aa, Artificial
  RAGE RD 5H3
DIQMTQSPSSLSASVGDRVTITCRASQDVGIYVNWFQQKPGKAPRRLIDRATNLADGVPSRFSGSRSGTDFTLTISSLQPEDFATYYCLEFDEHPLTFGGGTKVEIKDGGGSGGGGSGGGGSSEVLLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDITTGFDYWGQGTLVTVSS

SEQ ID NO: 12 - DNA, 726 bp, Artificial
  RAGE RD 10H6
gacatccagatgacccagtccccctcttctctgtctgcctctgtgggcgacggagtgaccatcacctgtcaggcctctcaggatgtgggcatctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggtggagatcaagaatggcggtggatcgggcggtggtggatctggaggaggtggaagctctgaggtgcagctggtggagtctggcggcggactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatcaccaccggcctcgactactggggccagggcaccctggtgaccgagtcctct

SEQ ID NO: 13 - DNA, 726 bp, Artificial
  RAGE RD 8G9
gacatccagatgacccagtccccctcttctctgtctgcctctgtgggcgacagagtgaccatcacctgtcgggcctctcaggatgtgggcatctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggtggagatcaaggatggcggtggatcgggcggtggtggatctggaggaggtggaagctctgaggtgcagctggtggagtctggcggcggactggtgcagcctggcagctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatcaccaccggcatcgactactggggccagggcaccctggtgaccgtgtcctct

SEQ ID NO: 14 - DNA, 726 bp, Artificial
  RAGE RD 10D8
gacatccagatgacccagtccccctcttctctgtctgcctctgtgggcgacaaagtgaccatcacctgtcgggcctcacaggatgtgggcttctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggtggagatcaaggatggcggtggatcgggcaatggtggatctggaggaggtggaagctctgaggtgcagctggtggagtctggcggtgaactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatcaccaccggcttcgactactggggccagggcaccctggtgaccgtgtcctct

SEQ ID NO: 15 - DNA, 726 bp, Artificial
  RAGE RD 3B3
gacatccagatgacccagtccccctcttctctgtctgcctctgtgggcgacagagtgaccatcacctgtcgggcctctcaggatgtgggcttctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaagatttcgccacctactactgcctggagttcgacgagcaccctctgacctttggcggcggaacaaaggtggagatcaaggatggcggtggatcgggcggtggtggatctggaggaggtggaagctctgaggtgcagctggtggagtctggcggcggactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatcaccaccggcatcgactactggggccagggcaccctggtgaccgtgtcctct

SEQ ID NO: 16 - DNA, 726 bp, Artificial
  RAGE RD 1G6
gacatccagatgacccagtccccctcttctctgtctgcctctgtgggcgacagagtgaccatcacctgtcgggcctctcaggatgtgggcttctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcgggacaaaggtggagatcaaggatggcggtggatcgggcggtggtggatctggaggaggtggaagctctgaggtgcagctggtggagtctggcggcggactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatcaccaacggcttcgactactggggccagggcaccctggtgaccgtgtcctct

SEQ ID NO: 17 - DNA, 726 bp, Artificial
  RAGE RD 2E6
gacatccagatgacccagtccccctcttctctgtctgcctctgtgggcgacagagtgaccatcacctgccgggcctctcaggatgtgggcatctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggtggagatcaaggatggcggtggatcgggcggtggtggatctggaggaggtggaaactctgaggtgcagctggtggagtctggcgtcggactggtgcagcctggcggctctctgagactgtcttgtgccgcctcaggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatcaccaacggcctcgactactggggccagggcaccctggtgaccgtgtcctct

SEQ ID NO: 18 - DNA, 726 bp, Artificial
  RAGE RD 5A3
gacatccagatgacccagtccccctcttctctgtctgcctctgtgggcgacatagtgaccatcacctgtcgggcctctcaggatgtgggcatctacgtgaactggtttcagcagaagcctggcaaggctcccaggcgcctgatctaccgggccaccaacctggccgatggcgtgccttccagattctccggctctcgctctagcaccgatttcaccctgaccatctcctccctccagcctgaggatttcgccacctactactgcctggagttcgacgaacatcctctgacctttggcggcggaacaaaggtggagatcaaggatggcggtggatcgggcggtggtggatctggaggaggtggaagctctgaggtgcagctggtggagtctggcggcggactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctcaagatgaactccctgagggccgaggataccgccgtgtactactgtaccagaggcggcgatatcaccaccggcatcgactactggggccagggcaccctggtgaccgtgtcctct

SEQ ID NO: 19 - DNA, 726 bp, Artificial
  RAGE RD 10G10
gacatccagatgacccagtccccctcttctctgtctgcctctgtgggcgacagagtgaccatcacctgtcgggcctctcaggatgtgggcatctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaagggtggagatcaaggatggcggtggatcgggcggtggtggatctggaggaggtggaagctctgaggtgcagctggtggagtctggcggcggactggtgcagcctggcggctctctgagactgtcttgtgccgcctccggcttcaccttcaacaactactggatgagctgggtgaggcaggcccctggcaagggcctggagtgggtggcctccatcgacaactccggcgagaacacctactaccccgactccgtgaaggaccggttcaccatctccagggacaacgccaagaactccctgtacctccagatgaactccctgagggccgaggataccgccgtgtactactgtgccagaggcggcgatatcaccaccggcttcgactactggggccagggcaccctggtgaccgtgtcctct

SEQ ID NO: 20 - DNA, 726 bp, Artificial
  RAGE RD 5H3
gacatccagatgacccagtccccctcttctctgtctgcctctgtgggcgacagagtgaccatcacctgtcgggcctctcaggatgtgggcatctacgtgaactggtttcagcagaagcctggcaaggctcccaggcgcctgatcgaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggttgagatcaaggatggcggtggatcgggcggtggtggatctggaggaggtggaagctctgaggttctactggtggagtctggcggcggactggtgcagcctggcggctctctgagactgtcttgtgccgcctccggcttcaccttcaacaactactggatgacctgggtgaggcaggcccctggcaagggcctggagtgggttgcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatcaccaccggcttcgactactggggccagggcaccctggtgaccgtgtcctct

SEQ ID NO: 21 - protein, 242 aa, Artificial
  RAGE S1R3A2 PDM4H12 1A6
DIQMTQSPSSLSASVGDRVTITCRASQDVGIYVNWFQQKPGKAPRRLIYRATNLADGVPSRFSGSRSGTDFTLTISSLQPEDFATYYCLEFDEHPLTFGGGTKVEIKDGGGSGGGGSGGGGSSEVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDILVSLDVWGQGTLVTVSS

SEQ ID NO: 22 - protein, 242 aa, Artificial
  RAGE S1R3A1 PDM4H12 1B2
DIQMTQSPSSLSASVGDRVTITCRASQDVGIYVNWFQQKPGKAPRRLIYRATNLADGVPSRFSGSRSGTDFTLTISSLQPEDFATYYCLEFDEHPLTFGGGTKVEIKDGGGSGGGGSGGGGSSEVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDILVGLDYWGQGTLVTVSS

SEQ ID NO: 23 - protein, 242 aa, Artificial
  RAGE S1R3A1 PDM4H12 1B4
DIQMTQSPSSLSASVGDRVTITCRASQDVGIYVNWFQQKPGKAPRRLIYRATNLADGVPSRFSGSRSGTDFTLTISSLQPEDFATYYCLEFDEHPLTFGGGTKVEIKDGGGSGGGGSGGGGSSEVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDIREGLRYWGQGTLVTVSS

SEQ ID NO: 24 - protein, 242 aa, Artificial
  RAGE S1R3A1 PDM4H12 1D2
DIQMTQSPSSLSASVGDRVTITCRASQDVGIYVNWFQQKPGKAPRRLIYRATNLADGVPSRFSGSRSGTDFTLTISSLQPEDFATYYCLEFDEHPLTFGGGTKVEIKDGGGSGGGGSGGGGSSEVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDIRVSLDHWGQGTLVTVSS

SEQ ID NO: 25 - protein, 242 aa, Artificial
  RAGE S1R3A1 PDM4H12 1G5
DIQMTQSPSSLSASVGDRVTITCRASQDVGIYVNWFQQKPGKAPRRLIYRATNLADGVPSRFSGSRSGTDFTLTISSLQPEDFATYYCLEFDEHPLTFGGGTKVEIKDGGGSGGGGSGGGGSSEVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDILDSLPYWGQGTLVTVSS

SEQ ID NO: 26 - protein, 242 aa, Artificial
  RAGE S1R6A1 PDM4H12 1B2
DIQMTQSPSSLSASVGDRVTITCRASQDVGIYVNWFQQKPGKAPRRLIYRATNLADGVPSRFSGSRSGTDFTLTISSLQPEDFATYYCLEFDEHPLTFGGGTKVEIKDGGGSGGGGSGGGGSSEVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDIRVGLDVWGQGTLVTVSS

SEQ ID NO: 27 - protein, 242 aa, Artificial
  RAGE S1R6A1 PDM4H12 1B6
DIQMTQSPSSLSASVGDRVTITCRASQDVGIYVNWFQQKPGKAPRRLIYRATNLADGVPSRFSGSRSGTDFTLTISSLQPEDFATYYCLEFDEHPLTFGGGTKVEIKDGGGSGGGGSGGGGSSEVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDIIISLDWWGQGTLVTVSS

SEQ ID NO: 28 - protein, 242 aa, Artificial
  RAGE S1R6A1 PDM4H12 1C2
DIQMTQSPSSLSASVGDRVTITCRASQDVGIYVNWFQQKPGKAPRRLIYRATNLADGVPSRFSGSRSGTDFTLTISSLQPEDFATYYCLEFDEHPLTFGGGTKVEIKDGGGSGGGGSGGGGSSEVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDIKVSLDRWGQGTLVTVSS

SEQ ID NO: 29 - protein, 242 aa, Artificial
  RAGE S1R6A1 PDM4H12 1C3
DIQMTQSPSSLSASVGDRVTITCRASQDVGIYVNWFQQKPGKAPRRLIYRATNLADGVPSRFSGSRSGTDFTLTISSLQPEDFATYYCLEFDEHPLTFGGGTKVEIKDGGGSGGGGSGGGGSSEVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDITLGLDVWGQGTLVTVSS

SEQ ID NO: 30 - protein, 242 aa, Artificial
  RAGE S1R6A1 PDM4H12 1G4
DIQMTQSPSSLSASVGDRVTITCRASQDVGIYVNWFQQKPGKAPRRLIYRATNLADGVPSRFSGSRSGTDFTLTISSLQPEDFATYYCLEFDEHPLTFGGGTKVEIKDGGGSGGGGSGGGGSSEVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDILDSLHFWGQGTLVTVSS

SEQ ID NO: 31 - DNA, 726 bp, Artificial
  RAGE S1R3A2 PDM4H12 1A6
gacatccagatgacccagtccccctcttctctgtctgcctctgtgggcgacagagtgaccatcacctgtcgggcctctcaggatgtgggcatctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggtggagatcaaggatggcggtggatcgggcggtggtggatctggaggaggtggaagctctgaggtgcagctggtggagtctggcggcggactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatcttggtgtccttggacgtgtggggccagggcaccctggtgaccgtgtcctct

SEQ ID NO: 32 - DNA, 726 bp, Artificial
  RAGE S1R3A1 PDM4H12 1B2
gacatccagatgacccagtccccctcttctctgtctgcctctgtgggcgacagagtgaccatcacctgtcgggcctctcaggatgtgggcatctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggtggagatcaaggatggcggtggatcgggcggtggtggatctggaggaggtggaagctctgaggtgcagctggtggagtctggcggcggactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatcctcgtggggctcgactactggggccagggcaccctggtgaccgtgtcctct

SEQ ID NO: 33 - DNA, 726 bp, Artificial
  RAGE S1R3A1 PDM4H12 1B4
gacatccagatgacccagtccccctcttctctgtctgcctctgtgggcgacagagtgaccatcacctgtcgggcctctcaggatgtgggcatctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggtggagatcaaggatggcggtggatcgggcggtggtggatctggaggaggtggaagctctgaggtgcagctggtggagtctggcggcggactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatccgggaggggctcaggtactggggccagggcaccctggtgaccgtgtcctct

SEQ ID NO: 34 - DNA, 726 bp, Artificial
  RAGE S1R3A1 PDM4H12 1D2
gacatccagatgacccagtccccctcttctctgtctgcctctgtgggcgacagagtgaccatcacctgtcgggcctctcaggatgtgggcatctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggtggagatcaaggatggcggtggatcgggcggtggtggatctggaggaggtggaagctctgaggtgcagctggtggagtctggcggcggactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatccgcgtgtcgttggaccactggggccagggcaccctggtgaccgtgtcctct

SEQ ID NO: 35 - DNA, 726 bp, Artificial
  RAGE S1R3A1 PDM4H12 1G5
gacatccagatgacccagtccccctcttctctgtctgcctctgtgggcgacagagtgaccatcacctgtcgggcctctcaggatgtgggcatctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggtggagatcaaggatggcggtggatcgggcggtggtggatctggaggaggtggaagctctgaggtgcagctggtggagtctggcggcggactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatcttggacagcttgccctactggggccagggcaccctggtgaccgtgtcctct

SEQ ID NO: 36 - DNA, 726 bp, Artificial
  RAGE S1R6A1 PDM4H12 1B2
gacatccagatgacccagtccccctcttctctgtctgcctctgtgggcgacagagtgaccatcacctgtcgggcctctcaggatgtgggcatctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggtggagatcaaggatggcggtggatcgggcggtggtggatctggaggaggtggaagctctgaggtgcagctggtggagtctggcggcggactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatccgggtggggttggacgtgtggggccagggcaccctggtgaccgtgtcctct

SEQ ID NO: 37 - DNA, 726 bp, Artificial
  RAGE S1R6A1 PDM4H12 1B6
gacatccagatgacccagtccccctcttctctgtctgcctctgtgggcgacagagtgaccatcacctgtcgggcctctcaggatgtgggcatctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggtggagatcaaggatggcggtggatcgggcggtggtggatctggaggaggtggaagctctgaggtgcagctggtggagtctggcggcggactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatcatcatctccttggactggtggggccagggcaccctggtgaccgtgtcctct

SEQ ID NO: 38 - DNA, 726 bp, Artificial
  RAGE S1R6A1 PDM4H12 1C2
gacatccagatgacccagtccccctcttctctgtctgcctctgtgggcgacagagtgaccatcacctgtcgggcctctcaggatgtgggcatctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggtggagatcaaggatggcggtggatcgggcggtggtggatctggaggaggtggaagctctgaggtgcagctggtggagtctggcggcggactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatcaaggtctccttggacaggtggggccagggcaccctggtgaccgtgtcctct

SEQ ID NO: 39 - DNA, 726 bp, Artificial
  RAGE S1R6A1 PDM4H12 1C3
gacatccagatgacccagtccccctcttctctgtctgcctctgtgggcgacagagtgaccatcacctgtcgggcctctcaggatgtgggcatctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggtggagatcaaggatggcggtggatcgggcggtggtggatctggaggaggtggaagctctgaggtgcagctggtggagtctggcggcggactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatcaccctcggcttggacgtctggggccagggcaccctggtgaccgtgtcctct

SEQ ID NO: 40 - DNA, 726 bp, Artificial
  RAGE S1R6A1 PDM4H12 1G4
gacatccagatgacccagtccccctcttctctgtctgcctctgtgggcgacagagtgaccatcacctgtcgggcctctcaggatgtgggcatctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggtggagatcaaggatggcggtggatcgggcggtggtggatctggaggaggtggaagctctgaggtgcagctggtggagtctggcggcggactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatcctggactcgttgcacttctggggccagggcaccctggtgaccgtgtcctct

SEQ ID NO: 41 - protein, 16 aa, Artificial
  Flexible Linker
DGGGSGGGGSGGGGSS

SEQ ID NO: 42 - protein, 11 aa, Artificial
  Invariant CDR1-Kappa sequences
XASXXXGXXXX

SEQ ID NO: 43 - protein, 9 aa, Artificial
  Invariant CDR3-Kappa
XEXXXXPXX

SEQ ID NO: 44 - protein, 17 aa, Artificial
  Invariant CDR2-H chain sequences
XXXXXXXXXYXXXXXXX

SEQ ID NO: 45 - protein, 10 aa, Artificial
  Invariant CDR3-H chain sequences
GGDIXXXXXX

SEQ ID NO: 46 - protein, 11 aa, Artificial
  CDR-1 Kappa Variants
XASXXXGXXXX

SEQ ID NO: 47 - protein, 17 aa, Artificial
  CDR2-H
XXXXXXXXXYXXXXXXX

SEQ ID NO: 48 - protein, 11 aa, Artificial
  CDR3-H
GGDIXXXXXXX

SEQ ID NO: 49 - protein, 16 aa, Artificial
  Flexible Linker
XGGGSGXGGSGGGGXS

SEQ ID NO: 50 - protein, 119 aa, Artificial
  10H6 VH
EVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDITTGLDYWGQGTLVTESS

SEQ ID NO: 51 - protein, 119 aa, Artificial
  8G9 VH
EVQLVESGGGLVQPGSSLRLSCAASGFTFNNYWMTWVRQAPGKGLEWVASIDNSGDNTYYPDSVKDRFTISRDNAKNSLYLQMNSLRAEDTAVYYCARGGDITTGIDYWGQGTLVTVSS

SEQ ID NO: 52 - protein, 119 aa, Artificial
  10D8 VH
EVQLVESGGELVQPGGSLRLSCAASGFTFNNYWMTWVRQAPGKGLEWVASIDNSGDNTYYPDSVKDRFTISRDNAKNSLYLQMNSLRAEDTAVYYCARGGDITTGFDYWGQGTLVTVSS

SEQ ID NO: 53 - protein, 119 aa, Artificial
  3B3 VH
EVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDITTGIDYWGQGTLVTVSS

SEQ ID NO: 54 - protein, 119 aa, Artificial
  1G6 VH
EVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDITNGFDYWGQGTLVTVSS

SEQ ID NO: 55 - protein, 119 aa, Artificial
  2EG VH
EVQLVESGVGLVQPGGSLRLSCAASGFTFNNYWMTWVRQAPGKGLEWVASIDNSGDNTYYPDSVKDRFTISRDNAKNSLYLQMNSLRAEDTAVYYCARGGDITNGLDYWGQGTLVTVSS

SEQ ID NO: 56 - protein, 119 aa, Artificial
  5A3 VH
EVQLVESGGGLVQPGGSLRLSCAASGFTFNNYWMTWVRQAPGKGLEWVASIDNSGDNTYYPDSVKDRFTISRDNAKNSLYLKMNSLRAEDTAVYYCTRGGDITTGIDYWGQGTLVTVSS

SEQ ID NO: 57 - protein, 119 aa, Artificial
  10G10 VH
EVQLVESGGGLVQPGGSLRLSCAASGFTFNNYWMSWVRQAPGKGLEWVASIDNSGENTYYPDSVKDRFTISRDNAKNSLYLQMNSLRAEDTAVYYCARGGDITTGFDYWGQGTLVTVSS

SEQ ID NO: 58 - protein, 119 aa, Artificial
  5H3 VH
EVLLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDITTGFDYWGQGTLVTVSS

SEQ ID NO: 59 - protein, 119 aa, Artificial
  3A6 VH
EVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDILVSLDVWGQGTLVTVSS

SEQ ID NO: 60 - protein, 119 aa, Artificial
  3B2 VH
EVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDILVGLDYWGQGTLVTVSS

SEQ ID NO: 61 - protein, 119 aa, Artificial
  3B4 VH
EVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDIREGLRYWGQGTLVTVSS

SEQ ID NO: 62 - protein, 121 aa, Artificial
  3D2 VH
EVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDIRVSLDHISWGQGTLVTVSS

SEQ ID NO: 63 - protein, 119 aa, Artificial
  3G5 VH
EVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDILDSLPYWGQGTLVTVSS

SEQ ID NO: 64 - protein, 119 aa, Artificial
  6B2 VH
EVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDIRVGLDVWGQGTLVTVSS

SEQ ID NO: 65 - protein, 119 aa, Artificial
  6B6 VH
EVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDIIISLDWWGQGTLVTVSS

SEQ ID NO: 66 - protein, 119 aa, Artificial
  6C2 VH
EVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDIKVSLDRWGQGTLVTVSS

SEQ ID NO: 67 - protein, 119 aa, Artificial
  6C3 VH
EVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDITLGLDVWGQGTLVTVSS

SEQ ID NO: 68 - protein, 119 aa, Artificial
  6G4 VH
EVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDILDSLHFWGQGTLVTVSS

SEQ ID NO: 69 - protein, 107 aa, Artificial
  RAGE RD 10H6 VL
DIQMTQSPSSLSASVGDGVTITCQASQDVGIYVNWFQQKPGKAPRRLIYRATNLADGVPSRFSGSRSGTDFTLTISSLQPEDFATYYCLEFDEHPLTFGGGTKVEIK

SEQ ID NO: 70 - protein, 107 aa, Artificial
  RAGE RD 10D8 VL
DIQMTQSPSSLSASVGDKVTITCRASQDVGFYVNWFQQKPGKAPRRLIYRATNLADGVPSRFSGSRSGTDFTLTISSLQPEDFATYYCLEFDEHPLTFGGGTKVEIK

SEQ ID NO: 71 - protein, 107 aa, Artificial
  RAGE RD 3B3 VL
DIQMTQSPSSLSASVGDRVTITCRASQDVGFYVNWFQQKPGKAPRRLIYRATNLADGVPSRFSGSRSGTDFTLTISSLQPEDFATYYCLEFDEHPLTFGGGTKVEIK

SEQ ID NO: 72 - protein, 107 aa, Artificial
  RAGE RD 5A3 VL
DIQMTQSPSSLSASVGDIVTITCRASQDVGIYVNWFQQKPGKAPRRLIYRATNLADGVPSRFSGSRSSTDFTLTISSLQPEDFATYYCLEFDEHPLTFGGGTKVEIK

SEQ ID NO: 73 - protein, 107 aa, Artificial
  RAGE RD 10G10 VL
DIQMTQSPSSLSASVGDRVTITCRASQDVGIYVNWFQQKPGKAPRRLIYRATNLADGVPSRFSGSRSGTDFTLTISSLQPEDFATYYCLEFDEHPLTFGGGTRVEIK

SEQ ID NO: 74 - protein, 107 aa, Artificial
  RAGE RD 5H3 VL
DIQMTQSPSSLSASVGDRVTITCRASQDVGIYVNWFQQKPGKAPRRLIDRATNLADGVPSRFSGSRSGTDFTLTISSLQPEDFATYYCLEFDEHPLTFGGGTKVEIK

SEQ ID NO: 75 - protein, 242 aa, Artificial
  Parental (VL 2.11,VH V2.0) scFv
DIQMTQSPSSLSASVGDRVTITCRASQDVGIYVNWFQQKPGKAPRRLIYRATNLADGVPSRFSGSRSGTDFTLTISSLQPEDFATYYCLEFDEHPLTFGGGTKVEIKDGGGSGGGGSGGGGSSEVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDITTGFDYWGQGTLVTVSS

SEQ ID NO: 76 - protein, 11 aa, Artificial
  CDRL-1
XASQDVGXYVN

SEQ ID NO: 77 - protein, 7 aa, Artificial
  CDRL-2
RATNLAD

SEQ ID NO: 78 - protein, 9 aa, Artificial
  CDRL-3
LEFDEHPLT

SEQ ID NO: 79 - protein, 10 aa, Artificial
  CDRH-1
GFTFNNYWMT

SEQ ID NO: 80 - protein, 17 aa, Artificial
  CFDRH-2
SIDNSGXNTYYPDSVKD

SEQ ID NO: 81 - protein, 10 aa, Artificial
  CDRH-3
GGDIXXXXXX

(19) United States
(12) Patent Application Publication
(10) Pub. No.: US 2010/0143349 A1
(43) Pub. Date: Jun. 10, 2010

(54) HUMANIZED ANTI-RAGE ANTIBODY

(75) Inventors: Simon Evan Hufton, Hitchin (GB); William James Jonathan Finlay, Dublin (IE); Orla Margaret Cunningham, Dublin (IE); Alfredo Darmanin Sheehan, Dublin (IE); Xuemei Germaine, Carlow (IE); Matthew Allister Lambert, Dublin (IE)

Correspondence Address: WYETH LLC;PATENT LAW GROUP, 5 GIRALDA FARMS, MADISON, NJ, 07940, US

(73) Assignee: Wyeth, Madison, NJ (US)

(21) Appl. No.: 12/540,038

(22) Filed: Aug. 12, 2009

Related U.S. Application Data
(60) Provisional application No. 61/088,273, filed on Aug. 12, 2008.

(51) Int. Cl. A61K 39/395; C07K 16/28; C07H 21/04; A61P 31/04; A61P 35/00; A61P 29/00; A61P 19/02; A61P 1/00; A61P 9/00; A61P 15/00; A61P 25/00; A61P 27/02; A61P 3/10

(52) U.S. Cl. 424/133.1; 530/387.3; 530/389.1; 536/23.53

(57) ABSTRACT

Compositions comprising antigen binding polypeptides that bind specifically to Receptor For Advanced Glycation End-product (RAGE) and comprises: one or more complementarity determining regions (CDRs) with improved binding efficiency over a parental monoclonal antibody to RAGE are described. Antibodies containing the CDR's and methods of treating a RAGE-related disease or disorder comprising administering to the subject a therapeutically effective amount of the compositions of the invention are also provided.

### CROSS REFERENCE TO RELATED APPLICATIONS

This application claims priority from U.S. Provisional Application No. 61/088,273, filed Aug. 12, 2008 which is incorporated by reference in its entirety.

### TECHNICAL FIELD

The present invention generally relates to compositions comprising antigen-binding polypeptides, antibodies and fragments thereof that bind specifically to a receptor for advanced glycation endproducts (RAGE), to methods in which such compositions are administered to human patients and non-human mammals to treat or prevent RAGE-related diseases and disorders.

### BACKGROUND

The receptor for advanced glycation endproducts (RAGE) is a multi-ligand cell surface member of the immunoglobulin super-family. RAGE consists of an extracellular domain, a single membrane-spanning domain, and a cytosolic tail. The extracellular domain of the receptor consists of one V-type immunoglobulin domain followed by two C-type immunoglobulin domains. RAGE also exists in a soluble form (sRAGE). RAGE is expressed by many cell types, e.g., endothelial and smooth muscle cells, macrophages and lymphocytes, in many different tissues, including lung, heart, kidney, skeletal muscle and brain. Expression is increased in chronic inflammatory states such as rheumatoid arthritis and diabetic nephropathy. Although its physiologic function is unclear, it is involved in the inflammatory response and may have a role in diverse developmental processes, including myoblast differentiation and neural development.

RAGE is an unusual pattern-recognition receptor that binds several different classes of endogenous molecules leading to various cellular responses, including cytokine secretion, increased cellular oxidant stress, neurite outgrowth and cell migration. The ligands of RAGE include advanced glycation end products (AGE's), which form in prolonged hyperglycemic states. However, AGE's may be only incidental, pathogenic ligands. In addition to AGES, known ligands of RAGE include proteins having beta-sheet fibrils that are characteristic of amyloid deposits and pro-inflammatory mediators, including Sloo/calgranulins (e.g., S100A12, S100B, S100A8-A9), serum amyloid (SAA) (fibrillar form), beta-Amyloid protein (Aβ), and high mobility group box-1 chromosomal protein 1 (HMGB1, also known as amphoterin). HMGB-1 has been shown to be a late mediator of lethality in two models of murine sepsis, and interaction between RAGE and ligands such as HMGB1 is believed to play an important role in the pathogenesis of sepsis and other inflammatory diseases.

A number of significant human disorders are associated with an increased production of ligands for RAGE or with increased production of RAGE itself. Consistently effective therapeutics are not available for many of these disorders. These disorders include, for example, many chronic inflammatory diseases, including rheumatoid and psoriatic arthritis and inflammatory bowel disease, cancers, diabetes and diabetic nephropathy, amyloidoses, cardiovascular diseases and sepsis. It would be beneficial to have safe and effective treatments for such RAGE-related disorders.

Sepsis is a systemic inflammatory response (SIRS) to infection, and remains a profound outcome in even previously normal patients. Sepsis is defined by the presence of at least 2 of the 4 clinical signs: hypo- or hyperthermia, tachycardia, tachypnea, hyperventilation, or abnormal leukogram. Sepsis with one organ dysfunction/failure is defined as severe sepsis, and severe sepsis with intractable hypotension is septic shock. Additional types of sepsis include septicemia and neonatal sepsis. More than 2 million cases of sepsis occur each year in the U.S., Europe, and Japan, with estimated annual costs of $17 billion and mortality rates ranging from 20-50%. In patients surviving sepsis, the intensive care unit (ICU) stay is extended on average by 65% compared to ICU patients not experiencing sepsis.

Despite recent market entries and continually improving hospital care, sepsis remains a significant unmet medical need. Treatment of septic patients is time and resource intensive. The syndrome continues to exhibit a 20-50% mortality rate. Safe and well-tolerated therapeutic agents that could reduce the progression from early sepsis to severe sepsis or septic shock, and thereby improve survival, could provide a break-through in sepsis therapy.

The Receptor for Advanced Glycation End-products (RAGE) is a multi-ligand member of the immunoglobulin superfamily of cell surface molecules. RAGE can be activated by a number of pro-inflammatory ligands including advanced glycoxidation end products (AGEs), S100/calgranulins, high mobility group box 1 protein (HMGB1), amyloid beta-peptides and the family of beta-sheet fibrils. RAGE, and several of its ligands, are implicated in a variety of acute and chronic diseases including atherosclerosis, diabetes, diabetic nephropathy, Alzheimer's disease, Multiple Sclerosis, rheumatoid arthritis, inflammatory bowel disease, sepsis and some cancers.

Anti-RAGE antibodies have been shown to have in vivo function in the murine cecal ligation and puncture (CLP) model of sepsis.

Additional anti-RAGE antibodies having improved potency may be useful for treatment of various indications, in addition to further treatment for sepsis.

### SUMMARY

In one aspect, the invention provides an isolated antigen binding polypeptide that binds specifically to Receptor For Advanced Glycation End-product (RAGE) and comprises one or more complementarity determining regions (CDRs) selected from the group consisting of: SEQ ID NO's 42 to 46, wherein said antigen binding polypeptide binds to human or mouse RAGE and in some embodiments competes for binding to human or mouse RAGE with antibody XT-M4. In some embodiments, the antigen binding peptide comprises one or more CDRs selected from the group consisting of: SEQ ID NO's 76 to 81.

In some embodiments, the invention provides an antigen binding polypeptide that further comprises at least one of a variable light chain domain (V_{L}) that is a variant of SEQ ID NO:1 and a variable heavy chain domain (V_{H}) that is a variant of SEQ ID NO:2, wherein the variant V_{L} comprises invariant framework residues at Q6, P8, S10, of Framework 1, P44, I48 of Framework 2, R61, S65, E81, C88 of Framework 3, and G99 of Framework 4 and the V_{H} variant comprises invariant framework residues S7, A24 of Framework 1 and V37 and Q39 and of Framework 2. The antigen binding polypeptides can comprise at least one of the CDR's selected from the group represented in SEQ ID NO.'s 46- to 48 and 76-81. In some embodiments, the antigen binding polypeptide further comprises at least one of a V_{L} and a V_{H}, wherein the V_{L} comprises SEQ ID NO:1 and the V_{H} comprises SEQ ID NO:2, with one or more framework substitutions selected from the group consisting of: R18K, R18G, R18I or R185; Y49D; G68S; K103R, K103E, K103N, K103Q, K103D, K103H, K103W and K103Y in SEQ ID NO:1 and Q3L, Q3H or Q3R, Q3K, Q3W or Q3Y; G9V or G9D; G10E; G16S; T35S; Q82K; Q82R, Q82H, Q82W or Q82Y; A97T or A97S; and V117E, V117L, V117M in SEQ ID NO:2.

The antigen binding polypeptide of the invention can be selected from the group consisting of a chimeric antibody, a humanized antibody, a human antibody, a single chain antibody, a tetrameric antibody, a tetravalent antibody, a multispecific antibody, a domain-specific antibody, a domain-deleted antibody, a fusion protein, an ScFc fusion protein, an Fab fragment, an Fab′ fragment, an F(ab′)2 fragment, an Fv fragment, an ScFv fragment, an Fd fragment, a single domain antibody, a dAb fragment; a small modular immunopharmaceutical (SMIP) a nanobody, a shark variable IgNAR domain or portions thereof, a CDR3 peptide, and a constrained FR3-CDR3-FR4 peptide.

In some embodiments, the antigen binding polypeptides bind to human RAGE with a dissociation constant (Kd) in the range of from at least about 1×10-7 M to about 1×10-10 M.

In some embodiments, the antigen binding polypeptides bind to human RAGE the polypeptide binds to the V domain of human RAGE.

In some embodiments, the antigen binding polypeptides bind to cells expressing RAGE in vitro and or in vivo.

In some embodiments, the antigen binding polypeptides bind to RAGE and inhibits the binding of a RAGE binding partner (RAGE-BP) to the RAGE.

In some embodiments, the antigen binding polypeptides can reduce or prevent binding of antibody XT-M4 to RAGE.

In some embodiments, the antigen binding polypeptide comprises a V_{L} amino acid sequence selected from the group consisting of: SEQ ID NO:'s. 69 through 74 and or a V_{H} amino acid sequence selected from the group consisting of: SEQ ID NO:'s. 50 through 68.

The antigen binding polypeptides can also comprise at least one mutation of an amino acid in the V_{L} or V_{H} that removes a glycosylation site and or a flexible peptide linker between the V_{H} and the V_{L} chain domains such as, e.g., when the antigen binding polypeptide comprises a VL and VH domain from an antibody as part of a fusion protein that is an ScFv.

In some embodiments, the antigen binding polypeptide comprises in sequential order from the amino to carboxy end a V_{L} domain, the flexible linker and a V_{H} domain. In other embodiments, the antigen binding polypeptide comprises in sequential order from the amino to carboxy end a V_{H} domain, the flexible linker and a V_{L} domain. In some embodiments, the invention comprises an antigen binding polypeptide having a flexible linker that comprises the linker of SEQ ID NO. 41, optionally having at least one substitution selected from the group consisting of: D1N, G7N S15N (as in SEQ ID NO:49).

In some embodiments, the antigen binding polypeptide comprises a V_{L} amino acid sequence that is at least 90% identical to SEQ ID NO: 1 and or a V_{H} amino acid sequence that is at least 90% identical to SEQ ID NO:2.

In some embodiments, the antigen binding polypeptides of the invention comprise an amino acid sequence of any of SEQ ID NOS 3-11 and 21-30. In some embodiments the invention provides an isolated nucleic acid encoding an antigen binding polypeptide as described herein. In some embodiments the antigen binding polypeptides are encoded by a nucleic acid sequence of any one of the nucleic acid sequences that are selected from the group consisting of SEQ ID NOs 12-20 and 31-40.

In some embodiments, the antigen binding polypeptide comprises an antibody or a fragment of an antibody. The antibody or fragment of an antibody can comprise a V_{H} amino acid sequence selected from the group consisting of: SEQ ID NO;'s 50 to 68 and or a V_{L} amino acid sequence selected from the group consisting of: SEQ ID NO:'s 69 to 74.

In some embodiments, the antigen binding polypeptide comprises at least one mutation of an amino acid in the V_{L} or V_{H} that removes a glycosylation site.

In some embodiments, the invention provides a pharmaceutical composition comprising an antibody or antibody fragment as described herein and a pharmaceutically acceptable excipient.

In some embodiments, the invention provides an isolated nucleic acid comprising a nucleotide sequence encoding an anti-RAGE antibody variable region amino acid sequence selected from the group consisting of SEQ ID NO;'s 51 through 75, and or an isolated nucleic acid that specifically hybridizes to a nucleic acid of SEQ ID NO;'s 51 through 75 or a complement thereof under stringent hybridization conditions. In some embodiments, the invention provides an isolated nucleic acid selected from the group consisting of SEQ ID NO:'s 12-20 and 31-40. In various embodiments, the invention provides recombinant nucleic acid vectors comprising a nucleic acid selected from the group consisting of SEQ ID NO:'s 12-20 and 31-40. In various embodiments, the invention provides recombinant cells containing nucleic acid vectors comprising a nucleic acid selected from the group consisting of SEQ ID NO:'s 12-20 and 31-40.

In some embodiments, the invention provides a method of treating a subject having a RAGE-related disease or disorder comprising administering to the subject a therapeutically effective amount an antigen binding polypeptide or pharmaceutical composition as described herein.

In various embodiments, the method of treating a RAGE-related disease or disorder comprises administering to the subject a composition comprising an antibody or antibody fragment that comprises a V_{H} domain comprising an amino acid sequence of any of SEQ ID NO:'s 50 to 68 and or a V_{L} domain comprising an amino acid sequence of any of SEQ ID NO:'s 69 to 74.

In various embodiments, the invention provides treatment of a RAGE-related disease or disorder that is selected from the group consisting of sepsis, septic shock, listeriosis, inflammatory diseases, cancers, arthritis, Crohn's disease, chronic acute inflammatory diseases, cardiovascular diseases, erectile dysfunction, diabetes, complications of diabetes, vasculitis, nephropathies, retinopathies, and neuropathies. In some embodiments, the compositions and methods of the invention can additionally comprise combining the composition or method with an additional one or more agents useful in the treatment of the RAGE-related disease or disorder that is to be treated. In some embodiments, the additional the agent is selected from the group consisting of: anti-inflammatory agents, antioxidants, beta-blockers, antiplatelet agents, ACE inhibitors, lipid-lowering agents, anti-angiogenic agents, and chemotherapeutics.

In some embodiments, the antigen binding polypeptide, antibody or antibody fragment of the invention specifically binds to soluble RAGE (sRAGE). In some embodiments, the antigen binding polypeptide, antibody or antibody fragment specifically binds to mouse and or human RAGE.

In some embodiments, the antigen binding polypeptide, antibody or antibody fragment specifically binds to sRAGE with a dissociation constant (Kd) in the range of from about 1×10-9 M to about 5×10-9 M.

In some embodiments, the invention provides the use of an antigen binding polypeptide, antibody or antibody fragment as described herein in the manufacture of a medicament for treating a RAGE related disorder in a subject, which subject can be a mammal and which subject can be a human subject.

### BRIEF DESCRIPTION OF THE DRAWINGS

FIG. 1 represents the amino acid sequence for SEQ ID NO:'s 1 and 2, which correspond to the parental V_{H}-chain and V_{L} chain of antibody XT-M4 (V_{H} V2.0, V_{L} 2.11). CDR's are indicated.

FIG. 2 represents results of initial screening for increased RAGE binding of scFV ribosome display clones.

FIG. 3 represents results of initial screening for increased RAGE binding of scFv phage display clones.

FIG. 4A-D represents results of HTRF analysis of recombinant scFv-Fc fusions binding to murine and human RAGE.

FIGS. 5A and B represents results of HTRF analysis of recombinant scFv-Fc fusions binding to human soluble RAGE.

FIGS. 6A and B represents results of ELISA binding of recombinant scFv-Fc fusions to RAGE expressed on CHO cells.

### SUMMARY OF THE SEQUENCE LISTING

SEQ ID NO:1 represents the amino acid sequence of parental humanized XT-M4 heavy chain domain.

SEQ ID NO:2 represents the amino acid sequence of parental XT-M4 light chain domain.

SEQ ID NO:'s 3-11 represent amino acid sequences of single chain fragments of variable region domains (scFv) having substituted heavy and light chains From XT-M4 that were selected and improved binding to RAGE.

SEQ ID NO:'s 12-20 represent nucleic acid sequences of single chain fragments of variable region domains (scFv) having substituted heavy and light chains from XT-M4 that were selected and improved binding to RAGE.

SEQ ID NO:'s 21-30 represent amino acid sequences of single chain fragments of variable region domains (scFv) having substituted heavy and light chains from XT-M4 that were selected and improved binding to RAGE.

SEQ ID NO:'s 31-40 represent nucleic acid sequences of single chain fragments of variable region domains (scFv) having substituted heavy and light chains from XT-M4 that were selected following ribosome display mutation and improved binding to RAGE.

SEQ ID NO:41 represents an exemplary linker amino acid sequences for use in scFv's.

SEQ ID NO:'s 42-48 represent amino acid sequences for complementarity determining regions (CDRs) selected for improved scFv binding to RAGE compared to an XT-M4 scFv.

SEQ ID NO:49 represents substitutions in linker amino acid sequences from scFvs selected for improved scFv binding to RAGE compared to an XT-M4 scFv.

SEQ ID NO:'s 50-68 represent amino acid sequences for V_{H} chains from scFv's selected for improved scFv binding to RAGE compared to humanized XT-M4 XT-M4 scFv.

SEQ ID NO:'s 69-74 represent amino acid sequences for V_{L} chains from scFv's selected for improved scFv binding to RAGE compared to humanized XT-M4 scFv.

SEQ ID NO:75 represents the amino acid sequence of parental XT-M4 scFv fusion V_{H} 2.0, V_{L} 2.11.

SEQ ID NO:'s 76-81 represent observed CDR sequences observed in ScFv's selected following mutation and selection for RAGE-binding.

### DETAILED DESCRIPTION

An anti-RAGE antibody, XT-M4, has previously been described in U.S. Patent Publication No. 200710286858A1, including chimeric and humanized version. A particular scFv humanized variant of XT-M4 (i.e., V_{H} 2.0, V_{L} 2.11) was also described in U.S. 200710286858A1. The V_{H} 2.0 and V_{L} 2.11 sequences also have been described in U.S. 200710286858A1. Nineteen (19) scFv variants of anti RAGE XT-M4 (V_{H} 2.0, V_{L} 2.11) were generated using either ribosome display or phage display technologies. The scFv variants demonstrated improved potencies in the range of 5-60 fold in an HTRF competition assay with parental rat IgG. The potency gains were further improved for the majority of clones when the scFv's were reformatted to bivalent scFv-Fc fusions. The maximum improvement in potency as a bivalent scFv-Fc fusion was found to be 62.7 fold relative to the parental XT-M4 scFv-Fc fusion, as measured by 1050 in HTRF on human RAGE. This improvement was shown to be primarily due to improvements in off-rate of up to 69-fold as measured by BIAcore (described below). All clones generated retained binding, and in many cases had improved binding to mouse RAGE. Further analysis on cell surface expressed human RAGE confirmed scFv-fusion variants also had improved binding to the cell surface target. A number of key mutations have been identified which can be clearly correlated with improved potency (e.g., V_{H}-CDR3 F106L and V_{L}-CDR1 I31F). V_{H}-CDR3 F106L is a crucial mutation and was identified independently by both ribosome and phage display technologies using random and targeted mutagenesis strategies respectively.

The present inventive antibodies have improved potency compared to the anti-RAGE antagonist antibody XT-M4 for human RAGE while maintaining, or improving, cross reactivity to mouse RAGE. An improved potency should translate into an improved efficacy in vivo, reduced dose or frequency of dose. To this end, the parental antibody was used in competition HTRF assays to maintain the parental epitope while screening for improved efficacy.

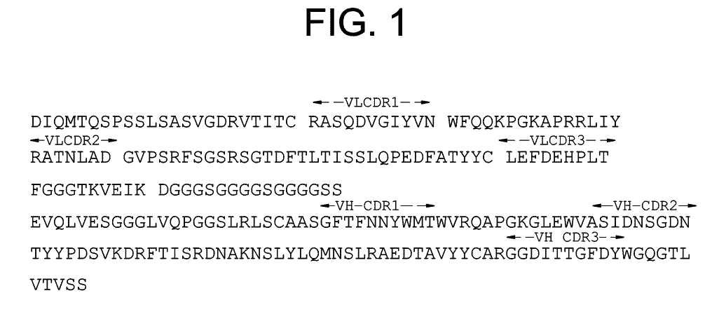

The starting antibody for optimization was the humanized variant of XT-M4 V_{H} 2.0, V_{L} 2.11. This variant of XT-M4 has 4 back mutations in the frameworks of the V_{L} domain (Y36F, K45R, L46R, G66R) and the putative N-linked glycosylation site in the V_{H}-CDR2 has been removed (N52D). The strategy taken was to use both phage display and ribosome display technologies to generate large libraries of mutated scFv antibody variants of a humanized XT-M4 from which improved clones would be isolated using antigen specific selection and screening procedures. The starting parental scFv antibody variant is shown in FIG. 1 and SEQ ID NO: 75 and the V_{L} and V_{H} domains are separately shown in SEQ ID NO:'s 1 (V_{L}) and 2 (V_{H}).

### DEFINITIONS

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The BIACORE™ method relies on the phenomenon of surface plasmon resonance (SPR), which occurs when surface plasmon waves are excited at a metal/liquid interface. Light is directed at, and reflected from, the side of the surface not in contact with sample, and SPR causes a reduction in the reflected light intensity at a specific combination of angle and wavelength. Bimolecular binding events cause changes in the refractive index at the surface layer, which are detected as changes in the SPR signal.

The term “or” is used herein to mean, and is used interchangeably with, the term “and/or,” unless context clearly indicates otherwise.

An “isolated” or “purified” polypeptide or protein, e.g., an “isolated antibody,” is purified to a state beyond that in which it exists in nature. For example, the “isolated” or “purified” polypeptide or protein, e.g., an “isolated antibody,” can be substantially free of cellular material or other contaminating proteins from the cell or tissue source from which the protein is derived, or substantially free from chemical precursors or other chemicals when chemically synthesized. The preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation. Proteins or polypeptides referred to herein as “recombinant” are proteins or polypeptides produced by the expression of recombinant nucleic acids.

The terms “antibody” or “antibody molecule” are used interchangeably with the term “immunoglobulin” herein, and includes intact antibodies, fragments of antibodies, e.g., Fab, F(ab′)₂ fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced IL 13 binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)₂, Fabc, Fd, dAb, and scFv and/or Fv fragments.

The term “antigen-binding fragment” or “antigen-binding polypeptide” can be used interchangeably to refer to a polypeptide fragment of an immunoglobulin, antibody or antibody-like molecule or other polypeptide molecule that binds antigen or competes with antibody that binds to the same antigenic site for antigen binding (i.e., specific binding). As such these antibodies or fragments thereof are included in the scope of the invention, provided that the antibody or fragment binds specifically to RAGE, and neutralizes or inhibits one or more RAGE-associated activities (e.g., inhibits binding of RAGE binding partners (RAGE-BPs) to RAGE).

The antibody includes a molecular structure comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

It is intended that the term “antibody” encompass any Ig class or any Ig subclass (e.g. the IgG₁, IgG₂, IgG₃, and IgG₄ subclasses of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG₁, IgG₂, IgG3, and IgG₄) that have been identified in humans and higher mammals. The antibodies can exist in monomeric or polymeric form; for example, IgM antibodies exist in pentameric form, and IgA antibodies exist in monomeric, dimeric or multimeric form.

The term “IgG subclass” refers to the four subclasses of immunoglobulin class IgG—IgG. 1, IgG₂, IgG₃, and IgG4 that have been identified in humans and higher mammals by the gamma. heavy chains of the immunoglobulins, Y1-Y4, respectively.

The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen.

The term “domain” refers to a globular region of a heavy or light chain polypeptide comprising peptide loops (e.g., comprising 3 to 4 peptide loops) stabilized, for example, by beta.-pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”.

The “constant” domains of an antibody light chain are referred to interchangeably as “light chain constant regions”, “light chain constant domains”, “CL” regions or “CL” domains.

The “constant” domains of an antibody heavy chain are referred to interchangeably as “heavy chain constant regions”, “heavy chain constant domains”, “CH” regions or “CH” domains).

The “variable” domains of an antibody light chain are referred to interchangeably as “light chain variable regions”, “light chain variable domains”, “VL” regions or “VL” domains).

The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

“Specific binding” of an antibody means that the exhibits appreciable affinity for a particular antigen or epitope and, generally, does not exhibit significant cross-reactivity. The term “anti-RAGE antibody” as used herein refers to an antibody that binds specifically to a RAGE. The antibody may exhibit no cross-reactivity (e.g., does not cross-react with non-RAGE peptides or with remote epitopes on RAGE. “Appreciable” binding includes binding with an affinity of at least 10⁶, 10⁷, 10⁸, 10⁹ M⁻¹, or 10¹⁰ M⁻¹. Antibodies with affinities greater than 10⁷ M⁻¹ or 10⁸ M⁻¹ typically bind with correspondingly greater specificity. Values intermediate of those set forth herein are also intended to be within the scope of the present invention and antibodies of the invention bind to RAGE with a range of affinities, for example, 10⁶ to 10¹⁰M⁻¹, or 10⁷ to 10¹⁰ M⁻¹, or 10⁸ to 10¹⁰ M⁻¹. An antibody that “does not exhibit significant cross-reactivity” is one that will not appreciably bind to an entity other than its target (e.g., a different epitope or a different molecule). For example, an antibody that specifically binds to RAGE will appreciably bind RAGE but will not significantly react with non-RAGE proteins or peptides. An antibody specific for a particular epitope will, for example, not significantly cross-react with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The BIACORE™ method relies on the phenomenon of surface plasmon resonance (SPR), which occurs when surface plasmon waves are excited at a metal/liquid interface. Light is directed at, and reflected from, the side of the surface not in contact with sample, and SPR causes a reduction in the reflected light intensity at a specific combination of angle and wavelength. Bimolecular binding events cause changes in the refractive index at the surface layer, which are detected as changes in the SPR signal.

The dissociation constant, Kd, and the association constant, Ka, are quantitative measures of affinity. At equilibrium, free antigen (Ag) and free antibody (Ab) are in equilibrium with antigen-antibody complex (Ag−Ab), and the rate constants, ka and kd, quantitate the rates of the individual reactions. At equilibrium, ka [Ab][Ag]=kd [Ag−Ab]. The dissociation constant, Kd, is given by: Kd=kd/ka=[Ag][Ab]/[Ag−Ab]. Kd has units of concentration, most typically M, mM, μM, nM, pM, etc. When comparing antibody affinities expressed as Kd, having greater affinity for RAGE is indicated by a lower value. The association constant, Ka, is given by: Ka=ka/kd=[Ag−Ab]/[Ag][Ab]. Ka has units of inverse concentration, most typically M⁻¹, mM⁻¹, μM⁻¹, nM⁻¹, pM¹, etc. As used herein, the term “avidity” refers to the strength of the antigen-antibody bond after formation of reversible complexes. Anti-RAGE antibodies may be characterized in terms of the Kd for their binding to a RAGE protein, as binding “with a dissociation constant (Kd) in the range of from about (lower Kd value) to about (upper Kd value).” In this context, the term “about” is intended to mean the indicated Kd value +−0.20%; i.e., Kd of about 1=Kd in the range of from 0.8 to 1.2.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The terms “binding portion” of an antibody (or “antibody portion”) or antigen-binding polypeptide include one or more complete domains, e.g., a pair of complete domains, as well as fragments of an antibody that retain the ability to specifically bind to an antigen, e.g., RAGE. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies (Muyldermans et al., 2001, 26:230-5), and an isolated complementarity determining region (CDR). Fab fragment is a monovalent fragment consisting of the VL, VH, CL and CH1 domains. F(ab′)₂ fragment is a bivalent fragment comprising two Fab fragments linked by a disulfide bridge at the hinge region. Fd fragment consists of the VH and CH1 domains, and Fv fragment consists of the VL and VH domains of a single arm of an antibody. A dAb fragment consists of a VH domain (Ward et al., (1989) Nature 341:544-546). While the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv) (Bird et al., 1988, Science 242:423-426). Such single chain antibodies are also intended to be encompassed within the term “binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, et al., 1993, Proc. Natl. Acad. Sci. USA 90:6444-6448). An antibody or binding portion thereof also may be part of a larger immunoadhesion molecules formed by covalent or non-covalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Binding fragments such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein and as known in the art. Other than “bispecific” or “bifunctional” antibodies, an antibody is understood to have each of its binding sites identical. A “bispecific” or “bifunctional antibody” is an artificial hybrid antibody having two different heavy/light chain pairs and two different binding sites. A bispecific antibody can also include two antigen binding regions with an intervening constant region. Bispecific antibodies can be produced by a variety of methods including fusion of hybridomas or linking of Fab′ fragments. See, e.g., Songsivilai et al., Clin. Exp. Immunol. 79:315-321, 1990; Kostelny et al., 1992, J. Immunol. 148, 1547-1553.

The term “backmutation” refers to a process in which some or all of the somatically mutated amino acids of a human antibody are replaced with the corresponding germline residues from a homologous germine antibody sequence. The heavy and light chain sequences of the human antibody of the invention are aligned separately with the germline sequences in the VBASE database to identify the sequences with the highest homology. Differences in the human antibody of the invention are returned to the germline sequence by mutating defined nucleotide positions encoding such different amino acid. The role of each amino acid thus identified as candidate for backmutation should be investigated for a direct or indirect role in antigen binding and any amino acid found after mutation to affect any desirable characteristic of the human antibody should not be included in the final human antibody; as an example, activity enhancing amino acids identified by the selective mutagenesis approach will not be subject to backmutation. To minimize the number of amino acids subject to backmutation those amino acid positions found to be different from the closest germline sequence but identical to the corresponding amino acid in a second germline sequence can remain, provided that the second germline sequence is identical and colinear to the sequence of the human antibody of the invention for at least 10, preferably 12 amino acids, on both sides of the amino acid in question. Backmutation may occur at any stage of antibody optimization; preferably, backmutation occurs directly before or after the selective mutagenesis approach. More preferably, backmutation occurs directly before the selective mutagenesis approach.

Intact antibodies, also known as immunoglobulins, are typically tetrameric glycosylated proteins composed of two light (L) chains of approximately 25 kDa each and two heavy (H) chains of approximately 50 kDa each. Two types of light chain, termed lambda and kappa, are found in antibodies. Depending on the amino acid sequence of the constant domain of heavy chains, immunoglobulins can be assigned to five major classes: A, D, E, G, and M, and several of these may be further divided into subclasses (isotypes), e.g., IgG1, IgG2, IgG3, IgG4, IgA1, and IgA2. Each light chain is composed of an N terminal variable (V) domain (VL) and a constant (C) domain (CL). Each heavy chain is composed of an N terminal V domain (VH), three or four C domains (CHs), and a hinge region. The CH domain most proximal to VH is designated as CH1. The VH and VL domains consist of four regions of relatively conserved sequences called framework regions (FR1, FR2, FR3, and FR4), which form a scaffold for three regions of hypervariable sequences (complementarity determining regions, CDRs). The CDRs contain most of the residues responsible for specific interactions of the antibody with the antigen. CDRs are referred to as CDR1, CDR2, and CDR3. Accordingly, CDR constituents on the heavy chain are referred to as H1, H2, and H3, while CDR constituents on the light chain are referred to as L1, L2, and L3. CDR3 is the greatest source of molecular diversity within the antibody-binding site. H3, for example, can be as short as two amino acid residues or greater than 26 amino acids. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988. One of skill in the art will recognize that each subunit structure, e.g., a CH, VH, CL, VL, CDR, FR structure, comprises active fragments, e.g., the portion of the VH, VL, or CDR subunit that binds to the antigen, i.e., the binding fragment, or, e.g., the portion of the CH subunit that binds to and/or activates, e.g., an Fc receptor and/or complement.

Antibody diversity is created by the use of multiple germline genes encoding variable regions and a variety of somatic events. The somatic events include recombination of variable gene segments with diversity (D) and joining (J) gene segments to make a complete VH region, and the recombination of variable and joining gene segments to make a complete VL region. The recombination process itself is imprecise, resulting in the loss or addition of amino acids at the V(D)J junctions. These mechanisms of diversity occur in the developing B-cell prior to antigen exposure. After antigenic stimulation, the expressed antibody genes in B-cells undergo somatic mutation. Based on the estimated number of germline gene segments, the random recombination of these segments, and random VH-VL pairing, up to 1.6.times.10⁷ different antibodies could be produced (Fundamental Immunology, 3rd ed. (1993), ed. Paul, Raven Press, New York, N.Y.). When other processes that contribute to antibody diversity (such as somatic mutation) are taken into account, it is thought that upwards of 1.times.10¹⁰ different antibodies could be generated (Immunoglobulin Genes, 2nd ed. (1995), eds. Jonio et al., Academic Press, San Diego, Calif.). Because of the many processes involved in generating antibody diversity, it is unlikely that independently derived monoclonal antibodies with the same antigen specificity will have identical amino acid sequences.

The term “dimerizing polypeptide” or “dimerizing domain” includes any polypeptide that forms a diner (or higher order complex, such as a trimer, tetramer, etc.) with another polypeptide. Optionally, the dimerizing polypeptide associates with other, identical dimerizing polypeptides, thereby forming homomultimers. An IgG Fc element is an example of a dimerizing domain that tends to form homomultimers. Optionally, the dimerizing polypeptide associates with other different dimerizing polypeptides, thereby forming heteromultimers. The Jun leucine zipper domain forms a dimer with the Fos leucine zipper domain, and is therefore an example of a dimerizing domain that tends to form heteromultimers. Dimerizing domains may form 25 both hetero- and homomultimers.

The term “human antibody” includes antibodies having variable and constant regions corresponding to human germline immunoglobulin sequences as described by Kabat et al. (See Kabat, et al. (1991) Sequences of proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. The mutations preferably are introduced using the “selective mutagenesis approach” described herein. The human antibody can have at least one position replaced with an amino acid residue, e.g., an activity enhancing amino acid residue, which is not encoded by the human germline immunoglobulin sequence. The human antibody can have up to twenty positions replaced with amino acid residues that are not part of the human germline immunoglobulin sequence. Further, up to ten, up to five, up to three or up to two positions are replaced. These replacements may fall within the CDR regions as described in detail below. However, the term “human antibody”, as used herein, is not intended to include antibodies in which CDR sequences derived from the germline of another mammalian species, such as a mouse, have been grafted onto human framework sequences.

The phrase “recombinant human antibody” includes human antibodies that are prepared, expressed, created or isolated by recombinant means, such as antibodies expressed using a recombinant expression vector transfected into a host cell (described further in Section II, below), antibodies isolated from a recombinant, combinatorial human antibody library (described further in Section III, below), antibodies isolated from an animal (e.g., a mouse) that is transgenic for human immunoglobulin genes (see e.g., Taylor, L. D., et al. (1992) Nucl. Acids Res. 20:6287-6295) or antibodies prepared, expressed, created or isolated by any other means that involves splicing of human immunoglobulin gene sequences to other DNA sequences.

Such recombinant human antibodies have variable and constant regions derived from human germline immunoglobulin sequences (See Kabat, E. A., et al. (1991) Sequences of Proteins of Immunological Interest, Fifth Edition, U.S. Department of Health and Human Services, NIH Publication No. 91-3242). However, such recombinant human antibodies may be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies may be the result of selective mutagenesis approach or backmutation or both.

An “isolated antibody” includes an antibody or antibody fragment that is substantially free of other antibodies having different antigenic specificities (e.g., an isolated antibody that specifically binds RAGE is substantially free of antibodies that specifically bind RAGE other than hRAGE). An isolated antibody that specifically binds RAGE may bind RAGE molecules from other species. Moreover, an isolated antibody may be substantially free of other cellular material and/or chemicals. HT term “isolated” has the same meaning when applied to an antibody-binding polypeptide.

A “neutralizing antibody” (or an “antibody that neutralizes RAGE activity”) includes an antibody whose binding to hRAGE results in modulation of the biological activity of hRAGE. This modulation of the biological activity of hRAGE can be assessed by measuring one or more indicators of hRAGE biological activity, such as inhibition of receptor binding in a human RAGE receptor binding assay. These indicators of hRAGE biological activity can be assessed by one or more of several standard in vitro or in vivo assays known in the art (see, e.g., Examples 6 and 7).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “activity” includes activities such as the binding specificity/affinity of an antibody for an antigen, for example, an anti-hRAGE antibody that binds to RAGE and/or the neutralizing potency of an antibody, for example, an anti-h RAGE antibody whose binding to hRAGE inhibits the biological activity of RAGE, e.g., inhibition of receptor binding in a human RAGE receptor binding assay.

An “expression construct” is any recombinant nucleic acid that includes an expressible nucleic acid and regulatory elements sufficient to mediate expression of the expressible nucleic acid protein or polypeptide in a suitable host cell.

The terms “fusion protein” and “chimeric protein” are interchangeable and refer to a protein or polypeptide that has an amino acid sequence having portions corresponding to amino acid sequences from two or more proteins. The sequences from two or more proteins may be full or partial (i.e., fragments) of the proteins. Fusion proteins may also have linking regions of amino acids between the portions corresponding to those of the proteins. Such fusion proteins may be prepared by recombinant methods, wherein the corresponding nucleic acids are joined through treatment with nucleases and ligases and incorporated into an expression vector.

The term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. The term “percent identical” or “percent identity” refers to sequence identity between two amino acid sequences or between two nucleotide sequences. Percent identity can be determined by comparing a position in each sequence that may be aligned for purposes of comparison. Expression as a percentage of identity refers to a function of the number of identical amino acids or nucleic acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g. default settings. ENTREZ is available through the National Center for Biotechnology Information, National Library of Medicine, National Institutes of Health, Bethesda, Md. The percent identity of two sequences may be determined by the GCG program with a gap weight of 1, e.g. each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Viols. 70: 173-187 (1997). Also, the GAP I program using the Needlenan and Wunsch alignment method can be utilized to align sequences. An alternative search strategy uses MPSRCH software, which runs on a MASPAR computer. MPSRCH uses a Smith-Waterman algorithm to score sequences 5 on a massively parallel computer. This approach improves the ability to pick up distantly related matches, and is especially tolerant of small gaps and nucleotide sequence errors. Nucleic acid-encoded amino acid sequences can be used to search both protein and DNA databases.

The terms “polypeptide” and “protein” are used interchangeably herein.

A “RAGE” protein is a “Receptor for Advanded Glycation End Products,” as known in the art. RAGE proteins include soluble RAGE (sRAGE) and endogenous secretory RAGE (esRAGE). Endogenous secretory RAGE is a RAGE splice variant that is released outside of the cells, where it is capable of binding AGE ligands and neutralizing AGE actions. See e.g., Koyama et al., ATVE, 2005; 25:2587-2593. Inverse association has been observed between human plasma esRAGE and several components of metabolic syndrome (BMI, insulin resistance, BP, hypertriglyceridemia and IGT). Plasma esRAGE levels have also been inversely associated with carotid and femoral atherosclerosis (quantitated by ultrasound) in subjects with or without diabetes. Moreover, plasma esRAGE levels are significantly lower in nondiabetic patients with angiographically proved coronary artery disease than age-matched healthy control.

A “Receptor for Advanced Glycation End Products Ligand Binding Element” or “RAGE-LBE” (also referred to herein as “RAGE-Fc” and “RAGE-strep”) includes any extracellular portion of a transmembrane RAGE polypeptide and fragments thereof that retain the ability to bind a RAGE ligand. This term also encompasses polypeptides having at least 85% identity, preferably at least 90% identity or more preferably at least 95% identity with a RAGE polypeptide, for example, the human or murine polypeptide to which a RAGE ligand or RAGE-BP will bind.

A “Receptor for Advanced Glycation End Products Binding Partner” or “RAGE-BP” includes any substance (e.g., polypeptide, small molecule, carbohydrate structure, etc.) that binds in a physiological setting to an extracellular portion of a RAGE protein (a receptor polypeptide such as, e.g., RAGE or RAGE-LBE).

“RAGE-related disorders” or “RAGE-associated disorders” include any disorder in which an affected cell or tissue exhibits an increase or decrease in the expression and/or activity of RAGE or one or more RAGE ligands. RAGE-related disorders also include any disorder that is treatable (i.e., one or more symptom may be eliminated or ameliorated) by a decrease in RAGE function (including, for example, administration of an agent that disrupts RAGE: RAGE-BP interactions).

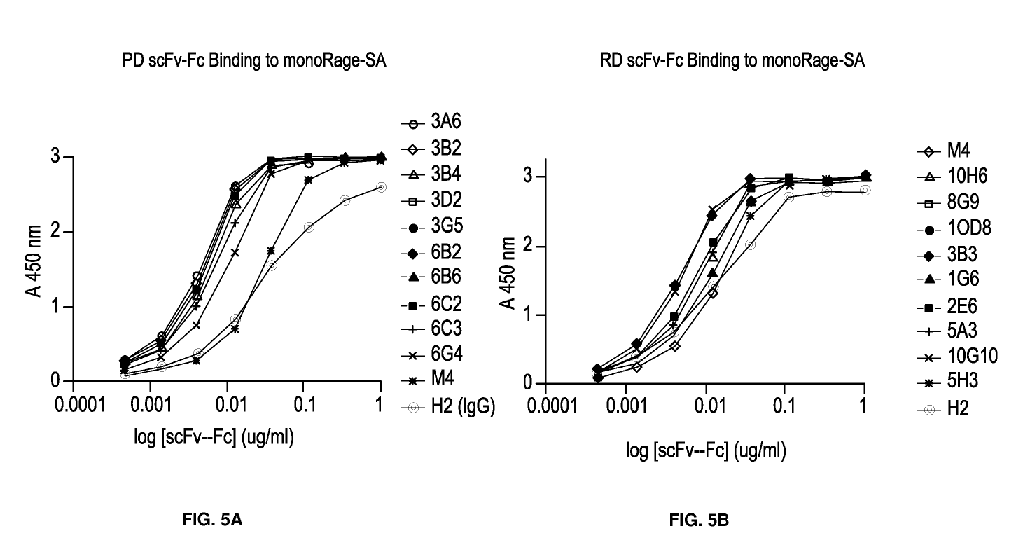

“V-domain of RAGE” refers to the immunoglobulin-like variable domain as shown in FIG. 5 of Neeper, et al, “Cloning and expression of RAGE: a cell surface receptor for advanced glycosylation end products of proteins,” J. Biol. Chem. 267:14998-15004 (1992), the contents of which are hereby incorporated by reference.

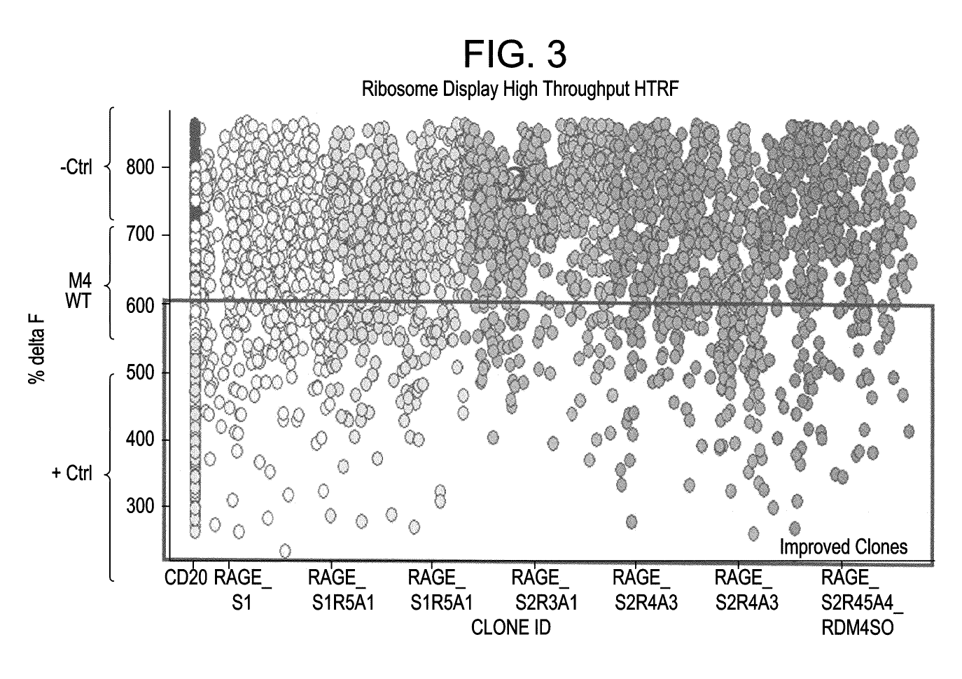

The human cDNA of RAGE is 1406 base pairs and encodes a mature protein of 404 amino acids. See FIG. 3 of Neeper et al. 1992.

The term “recombinant nucleic acid” includes any nucleic acid comprising at least two sequences that are not present together in nature. A recombinant nucleic acid may be generated in vitro, for example by using the methods of molecular biology, or in vivo, for example by insertion of a nucleic acid at a novel chromosomal location by homologous or non-homologous recombination.

The term “treating” with regard to a subject, refers to improving at least one symptom of the subject's disease or disorder. Treating can mean curing the disease or condition or improving it.

The term “vector” refers to a nucleic acid molecule capable of transporting another nucleic acid to which it has been linked. One type of vector is an episome, i.e., a nucleic acid capable of extra-chromosomal replication. Another type of vector is an integrative vector that is designed to recombine with the genetic material of a host cell. Vectors may be both autonomously replicating and integrative, and the properties of a vector may differ depending on the cellular context (i.e., a vector may be autonomously replicating in one host cell type and purely integrative in another host cell type). Vectors capable of directing the expression of expressible nucleic acids to which they are operatively linked are referred to herein as “expression vectors.”

“Specifically immunoreactive” or “specific binding” refer to the preferential binding of compounds [e.g., an antibody] to a particular peptide sequence, when an antibody interacts with a specific peptide sequence.

The phrase “effective amount” as used herein means that amount of one or more agent, material, or composition comprising one or more agents of the present invention that is effective for producing some desired effect in an animal. It is recognized that when an agent is being used to achieve a therapeutic effect, the actual dose which comprises the “effective amount” will vary depending on a number of conditions including the particular condition being treated, the severity of the disease, the size and health of the patient, the route of administration, etc. A skilled medical practitioner can readily determine the appropriate dose using methods well known in the medical arts.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline, (18) Ringer's solution, (19) ethyl alcohol; (20) phosphate buffer solutions; and (21) other non-toxic compatible substances employed in pharmaceutical formulations.

Humanization

Chimeric antibodies comprise sequences from at least two different species. As one example, recombinant cloning techniques may be used to include variable regions, which contain the antigen-binding sites, from a non-human antibody (i.e., an antibody prepared in a non-human species immunized with the antigen) and constant regions derived from a human immunoglobulin.

Humanized antibodies are a type of chimeric antibody wherein variable region residues responsible for antigen binding (i.e., residues of a complementarity determining region, abbreviated complementarity determining region, or any other residues that participate in antigen binding) are derived from a non-human species, while the remaining variable region residues (i.e., residues of the framework regions) and constant regions are derived, at least in part, from human antibody sequences. A subset of framework region residues and constant region residues of a humanized antibody may be derived from non-human sources. Variable regions of a humanized antibody are also described as humanized (i.e., a humanized light or heavy chain variable region). The non-human species is typically that used for immunization with antigen, such as mouse, rat, rabbit, non-human primate, or other non-human mammalian species. Humanized antibodies are typically less immunogenic than traditional chimeric antibodies and show improved stability following administration to humans. See e.g., Benincosa et al. (2000) J. Pharmacol. Exp. Ther. 292:810-6; Kalofonos et al. (1994) Eur. J. Cancer 30A:1842-50; Subramanian et al. (1998) Pediatr. Infect. Dis. J. 17:110-5.

Complementarity determining regions (CDRs) are residues of antibody variable regions that participate in antigen binding.

Several numbering systems for identifying CDRs are in common use. The Kabat definition is based on sequence variability, and the Chothia definition is based on the location of the structural loop regions. The AbM definition is a compromise between the Kabat and Chothia approaches. The CDRs of the light chain variable region are bounded by the residues at positions 24 and 34 (CDR1-L), 50 and 56 (CDR2-L), and 89 and 97 (CDR3-L) according to the Kabat, Chothia, or AbM algorithm. According to the Kabat definition, the CDRs of the heavy chain variable region are bounded by the residues at positions 31 and 35B (CDR1-H), 50 and 65 (CDR2-H), and 95 and 102 (CDR3-H) (numbering according to Kabat). According to the Chothia definition, the CDRs of the heavy chain variable region are bounded by the residues at positions 26 and 32 (CDR1-H), 52 and 56 (CDR2-H), and 95 and 102 (CDR3-H) (numbering according to Chothia). According to the AbM definition, the CDRs of the heavy chain variable region are bounded by the residues at positions 26 and 35B (CDR1-H), 50 and 58 (CDR2-H), and 95 and 102 (CDR3-H) (numbering according to Kabat). See Martin et al. (1989) Proc. Natl. Acad. Sci. USA 86: 9268-9272; Martin et al. (1991) Methods Enzymol. 203: 121-153; Pedersen et al. (1992) Immunomethods 1: 126; and Rees et al. (1996) In Sternberg M. J. E. (ed.), Protein Structure Prediction, Oxford University Press, Oxford, pp. 141-172.

A humanized antibody of the invention may be constructed to comprise one or more CDRs. Still further, CDR's may be used separately or in combination in synthetic molecules such as SMIPs and small antibody mimetics.

Specificity determining regions (SDRs) are residues within CDRs that directly interact with antigen. The SDRs correspond to hypervariable residues. See (Padlan et al. (1995) FASEB J. 9: 133-139).

Framework residues are those residues of antibody variable regions other than hypervariable or CDR residues. Framework residues may be derived from a naturally occurring human antibody, such as a human framework that is substantially similar to a framework region of the anti-RAGE antibody of the invention. Artificial framework sequences that represent a consensus among individual sequences may also be used. When selecting a framework region for humanization, sequences that are widely represented in humans may be preferred over less populous sequences. Additional mutations of the human framework acceptor sequences may be made to restore murine residues believed to be involved in antigen contacts and/or residues involved in the structural integrity of the antigen-binding site, or to improve antibody expression. A peptide structure prediction may be used to analyze the humanized variable heavy and light region sequences to identify and avoid post-translational protein modification sites introduced by the humanization design.

Humanized antibodies may be prepared using any one of a variety of methods including veneering, grafting of complementarity determining regions (CDRs), grafting of abbreviated CDRs, grafting of specificity determining regions (SDRs), and Frankenstein assembly, as described below. Humanized antibodies also include superhumanized antibodies, in which one or more changes have been introduced in the CDRs. For example, human residues may be substituted for non-human residues in the CDRs. These general approaches may be combined with standard mutagenesis and synthesis techniques to produce an anti-RAGE antibody of any desired sequence.

Veneering is based on the concept of reducing potentially immunogenic amino acid sequences in a rodent or other non-human antibody by resurfacing the solvent accessible exterior of the antibody with human amino acid sequences. Thus, veneered antibodies appear less foreign to human cells than the unmodified non-human antibody. See Padlan (1991) Mol. Immunol. 28:489-98. A non-human antibody is veneered by identifying exposed exterior framework region residues in the non-human antibody, which are different from those at the same positions in framework regions of a human antibody, and replacement of the identified residues with amino acids that typically occupy these same positions in human antibodies.

Grafting of CDRs is performed by replacing one or more CDRs of an acceptor antibody (e.g., a human antibody or other antibody comprising desired framework residues) with CDRs of a donor antibody (e.g., a non-human antibody). Acceptor antibodies may be selected based on similarity of framework residues between a candidate acceptor antibody and a donor antibody. For example, according to the Frankenstein approach, human framework regions are identified as having substantial sequence homology to each framework region of the relevant non-human antibody, and CDRs of the non-human antibody are grafted onto the composite of the different human framework regions. A related method also useful for preparation of antibodies of the invention is described in U.S. Patent Application Publication No. 2003/0040606.

Grafting of abbreviated CDRs is a related approach. Abbreviated CDRs include the specificity-determining residues and adjacent amino acids, including those at positions 27d-34, 50-55 and 89-96 in the light chain, and at positions 31-35b, 50-58, and 95-101 in the heavy chain (numbering convention of (Kabat et al. (1987)). See (Padlan et al. (1995) FASEB J. 9: 133-9). Grafting of specificity-determining residues (SDRs) is premised on the understanding that the binding specificity and affinity of an antibody combining site is determined by the most highly variable residues within each of the complementarity determining regions (CDRs). Analysis of the three-dimensional structures of antibody-antigen complexes, combined with analysis of the available amino acid sequence data may be used to model sequence variability based on structural dissimilarity of amino acid residues that occur at each position within the CDR. SDRs are identified as minimally immunogenic polypeptide sequences consisting of contact residues. See Padlan et al. (1995) FASEB J. 9: 133-139.

CDR's of the present description can also be utilized in small antibody mimetics, which comprise two CDR regions and a framework region (Qui et al. Nature Biotechnology Vol 25; 921-929; August 2007).

Acceptor frameworks for grafting of CDRs or abbreviated CDRs may be further modified to introduce desired residues. For example, acceptor frameworks may comprise a heavy chain variable region of a human sub-group I consensus sequence, optionally with non-human donor residues at one or more of positions 1, 28, 48, 67, 69, 71, and 93. As another example, a human acceptor framework may comprise a light chain variable region of a human sub-group I consensus sequence, optionally with non-human donor residues at one or more of positions 2, 3, 4, 37, 38, 45 and 60. Following grafting, additional changes may be made in the donor and/or acceptor sequences to optimize antibody binding and functionality. See e.g., PCT International Publication No. WO 91/09967.

Human frameworks of a heavy chain variable region that may be used to prepare humanized anti-RAGE antibodies include framework residues of DP-75, DP54, DP-54 FW VH 3 JH4, DP-54 VH3 3-07, DP-8 (VH1-2), DP-25, VI-2b and VI-3 (VH1-03), DP-15 and V1-8 (VH1-08), DP-14 and V1-18 (VH1-18), DP-5 and V1-24P (VH1-24), DP-4 (VH1-45), DP-7 (VH1-46), DP-10, DA-6 and YAC-7 (VH1-69), DP-88 (VH1-e), DP-3, and DA-8 (VH1-f).

Human frameworks of a light chain variable region that may be used to prepare humanized anti-RAGE antibodies include framework residues of human germ line clone DPK24, DPK-12, DPK-9 Vk1, DPK-9 Jk4, DPK9 Vk1 02, and germ line clone subgroups VkappaIII and VkappaI. The following mutations of a DPK24 germ line may increase antibody expression: F10S, T45K, 163S, Y67S, F73L, and T77S.

Representative humanized anti-RAGE antibodies of the invention include antibodies having one or more CDRs of a variable region amino acid sequence selected from SEQ ID NO:'s 42-48 and one or more VH and VL chain sequences selected from SEQ ID NO:'s 50-69.

Humanized anti-RAGE antibodies of the invention may be constructed wherein the variable region of a first chain (i.e., the light chain variable region or the heavy chain variable region) is humanized, and wherein the variable region of the second chain is not humanized (i.e., a variable region of an antibody produced in a non-human species). These antibodies are a type of humanized antibody referred to as semi-humanized antibodies.

The constant regions of chimeric and humanized anti-RAGE antibodies may be derived from constant regions of any one of IgA, IgD, IgE, IgG, IgM, and any isotypes thereof (e.g., IgG1, IgG2, IgG3, or IgG4 isotypes of IgG). The amino acid sequences of many antibody constant regions are known. The choice of a human isotype and modification of particular amino acids in the isotype may enhance or eliminate activation of host defense mechanisms and alter antibody biodistribution. See (Reff et al. (2002) Cancer Control 9: 152-66). For cloning of sequences encoding immunoglobulin constant regions, intronic sequences may be deleted.

Chimeric and humanized anti-RAGE antibodies may be constructed using standard techniques known in the art. For example, variable regions may be prepared by annealing together overlapping oligonucleotides encoding the variable regions and ligating them into an expression vector containing a human antibody constant region. See e.g., Harlow & Lane (1988) Antibodies: A Laboratory Manual, Cold Spring Harbor Laboratory Press, Cold Spring Harbor, N.Y. and U.S. Pat. Nos. 4,196,265; 4,946,778; 5,091,513; 5,132,405; 5,260,203; 5,677,427; 5,892,019; 5,985,279; 6,054,561. Tetravalent antibodies (H₄ L₄) comprising two intact tetrameric antibodies, including homodimers and heterodimers, may be prepared, for example, as described in PCT International Publication No. WO 02/096948. Antibody dimers may also be prepared via introduction of cysteine residue(s) in the antibody constant region, which promote interchain disulfide bond formation, by use of heterobifunctional cross-linkers (Wolff et al. (1993) Cancer Res. 53: 2560-5), or by recombinant production to include a dual constant region (Stevenson et al. (1989) Anticancer Drug Des. 3: 219-30). Antigen-binding fragments of antibodies of the invention may be prepared, for example, by expression of truncated antibody sequences, or by post-translation digestion of full-length antibodies.

Variants of anti-RAGE antibodies of the invention may be readily prepared to include various changes, substitutions, insertions, and deletions. For example, antibody sequences may be optimized for codon usage in the cell type used for antibody expression. To increase the serum half-life of the antibody, a salvage receptor binding epitope may be incorporated, if not present already, into the antibody heavy chain sequence. See U.S. Pat. No. 5,739,277. Additional modifications to enhance antibody stability include modification of IgG4 to replace the serine at residue 241 with proline. See Angal et al. (1993) Mol. Immunol. 30: 105-108. Other useful changes include substitutions as required to optimize efficiency in conjugating the antibody with a drug. For example, an antibody may be modified at its carboxyl terminus to include amino acids for drug attachment, for example one or more cysteine residues may be added. The constant regions may be modified to introduce sites for binding of carbohydrates or other moieties.

Additional antibody variants include glycosylation isoforms that result in improved functional properties. For example, modification of Fc glycosylation can result in altered effector functions, e.g., increased binding to Fc gamma receptors and improved ADCC and/or could decreased C1q binding and CDC (e.g., changing of Fc oligosaccharides from complex form to high-mannose or hybrid type may decrease C1q binding (Duncan and Winter ('88) Nature 322:738) and CDC (see, e.g., Kanda et al., Glycobiology, 2007:17:104-118)).

For example, mutations in the hinge region (e.g., at positions 234, 235, 236, and or 237) in all isotypes reduce affinity for Fcγ receptors, particular FcγR1 (Isaacs et al., (1998) J Immunol 161:3862-3869; Duncan et al. (1988) Nature 332:563; and Chappel et al. (1991) PNAS 88:9036 for FcR residues). Optionally, positions 234, 235, 236, and/or 237 can be substituted with alanine and 236 also with glutamine. A preferred combination for human IgG1 is L234A, L235A, and G237A. Other substitutions that decrease binding to Fc receptors is E233P and D265A (Shields et al. 2001, JBC 276: 6591-6604).

Modification also can be done by bioengineering bacteria, yeast, plant cells, insect cells, and mammalian cells; it can also be done by manipulating protein or natural product glycosylation pathways in genetically engineered organisms. Glycosylation can also be altered by exploiting the liberality with which sugar-attaching enzymes (glycosyltransferases) tolerate a wide range of different substrates. Finally, one of skill in the art can glycosylate proteins and natural products through a variety of chemical approaches: with small molecules, enzymes, protein ligation, metabolic bioengineering, or total synthesis. Examples of suitable small molecule inhibitors of N-glycan processing include, Castanospermine (CS), Kifunensine (KF), Deoxymannojirimycin (DMJ), Swainsonine (Sw), Monensin (Mn).

Variants of anti-RAGE antibodies of the invention may be produced using standard recombinant techniques, including site-directed mutagenesis, or recombination cloning. A diversified repertoire of anti-RAGE antibodies may be prepared via gene arrangement and gene conversion methods in transgenic non-human animals (U.S. Patent Publication No. 2003/0017534), which are then tested for relevant activities using functional assays. In particular embodiments of the invention, variants are obtained using an affinity maturation protocol for mutating CDRs (Yang et al. (1995) J. Mol. Biol. 254: 392-403), chain shuffling (Marks et al. (1992) Biotechnology (NY) 10: 779-783), use of mutator strains of E. coli (Low et al. (1996) J. Mol. Biol. 260: 359-368), DNA shuffling (Patten et al. (1997) Curr. Opin. Biotechnol. 8: 724-733), phage display (Thompson et al. (1996) J. Mol. Biol. 256: 77-88), and sexual PCR (Crameri et al. (1998) Nature 391: 288-291). For immunotherapy applications, relevant functional assays include specific binding to human RAGE antigen, antibody internalization, and targeting to a tumor site(s) when administered to a tumor-bearing animal, as described herein below.

The present invention further provides cells and cell lines expressing anti-RAGE antibodies and other antigen-binding polypeptides of the invention. Representative host cells include bacterial, yeast, mammalian and human cells, such as CHO cells, HEK-293 cells, HeLa cells, CV-1 cells, and COS cells. Methods for generating a stable cell line following transformation of a heterologous construct into a host cell are known in the art. Representative non-mammalian host cells include insect cells (Potter et al. (1993) Int. Rev. Immunol. 10(2-3):103-112). Antibodies may also be produced in transgenic animals (Houdebine (2002) Curr. Opin. Biotechnol. 13(6):625-629) and transgenic plants (Schillberg et al. (2003) Cell Mol. Life Sci. 60(3):433-45).

As discussed above, monoclonal, chimeric and humanized antibodies, which have been modified by, e.g., deleting, adding, or substituting other portions of the antibody, e.g., the constant region, are also within the scope of the invention. For example, an antibody can be modified as follows: (i) by deleting the constant region; (ii) by replacing the constant region with another constant region, e.g., a constant region meant to increase half-life, stability or affinity of the antibody, or a constant region from another species or antibody class; or (iii) by modifying one or more amino acids in the constant region to alter, for example, the number of glycosylation sites, effector cell function, Fc receptor (FcR) binding, complement fixation, among others.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. No. 5,624,821 and U.S. Pat. No. 5,648,260, the contents of all of which are hereby incorporated by reference). Similar type of alterations could be described which if applied to the murine, or other species immunoglobulin would reduce or eliminate these functions.

For example, it is possible to alter the affinity of an Fc region of an antibody (e.g., an IgG, such as a human IgG) for an FcR (e.g., Fc.gamma.R1), or for C1q binding by replacing the specified residue(s) with a residue(s) having an appropriate functionality on its side chain, or by introducing a charged functional group, such as glutamate or aspartate, or perhaps an aromatic non-polar residue such as phenylalanine, tyrosine, tryptophan or alanine (see e.g., U.S. Pat. No. 5,624,821). For example, substituting at least one of positions 318, 320, and/or 322 of the heavy chain with a residue having a different side chain can alter the affinity of an antibody for C1q. Other suitable alterations include 318 (Glu), 320 (Lys), and 322 (Lys) to Ala or other alkyl-substituted non-ionic residues such as Gly, Ile, Leu, or Val or such aromatic non-polar residues such as Phe, Tyr, Trp, and Pro. Polar non-ionic residues such as Ser, Thr, Cys, and Met can be substituted at positions 320 and 322. The antibody or binding fragment thereof may be conjugated with a cytotoxin, a therapeutic agent, or a radioactive metal ion. In one embodiment, the protein that is conjugated is an antibody or fragment thereof. A cytotoxin or cytotoxic agent includes any agent that is detrimental to cells. Non-limiting examples include, calicheamicin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, and analogs, or homologs thereof. Therapeutic agents include, but are not limited to, antimetabolites (e.g., methotrexate, 6-mercaptopurine, 6-thioguanine, cytarabine, and 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP), cisplatin), anthracyclines (e.g., daunorubicin and doxorubicin), antibiotics (e.g., dactinomycin, bleomycin, mithramycin, and anthramycin), and anti-mitotic agents (e.g., vincristine and vinblastine). Techniques for conjugating such moieties to proteins are well known in the art. Alternatively, it is now possible to produce transgenic animals (e.g., mice) that are capable, upon immunization, of producing a full repertoire of human antibodies in the absence of endogenous immunoglobulin production. For example, it has been described that the homogeneous deletion of the antibody heavy-chain joining region (JM) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jackobovits et al., Proc. Natl. Acad. Sci. USA, 90:2551 (1993); Jakobovits et al., Nature, 362:255-258 (1993); Bruggermann et al., Year in Immune, 7:33 (1983); and Duchosal et al. Nature 355:258 (1992). Human antibodies can also be derived from phage-display libraries (Hoogenboom et al., J. Mol. Biol. 227:381 (1991); Marks et al., J. Mol. Biol., 222:581-597 (1991); Vaughan et al. Nature Biotech 14:309 (1996)).

In certain embodiments, antibodies of the present invention can be administered in combination with other agents as part of a combinatorial therapy. For example, in the case of inflammatory conditions, the subject antibodies can be administered in combination with one or more other agents useful in the treatment of inflammatory diseases or conditions. In the case of cardiovascular disease conditions, and particularly those arising from atherosclerotic plaques, which are thought to have a substantial inflammatory component, the subject antibodies can be administered in combination with one or more other agents useful in the treatment of cardiovascular diseases. In the case of cancer, the subject antibodies can be administered in combination with one or more anti-angiogenic factors, chemotherapeutics, or as an adjuvant to radiotherapy. It is further envisioned that the administration of the subject antibodies will serve as part of a cancer treatment regimen that may combine many different cancer therapeutic agents. In the case of IBD, the subject antibodies can be administered with one or more anti-inflammatory agents, and may additionally be combined with a modified dietary regimen.

General Procedures

Optimization of antibodies can involve the improvement of one or more physical characteristics. Affinity of the antibody can be increased, specificity for target antigen (i.e., reduction of cross reactivity) can also be performed separately or concurrently with improved affinity.

Optimization can be explored via targeted or non-targeted mutagenesis of an antibody with desired characteristics. In targeted mutagenesis, specific areas known to be associated with affinity and specificity are preferentially mutated. VH-CDR3 is a primary target in this procedure as it is situated at the center of the antibody combining site and is the most naturally diverse loop in the immune repertoire. Targeted mutations can be incorporated into the VH-CDR3 or other loops like VL-CDR3 using “spiking” ‘parsimonious’ or “randomization” procedures which incorporate an increasing mutational load with the loop being targeted.

In non-targeted mutagenesis, random changes in all CDRs as well as framework regions were generated, followed by screening for antigen binding. In this manner random mutations were analyzed for improved antigen binding.

Some antibody molecules, e.g., Fabs, can be produced in bacterial cells, e.g., E. coli cells. For example, if the Fab is encoded by sequences in a phage display vector that includes a suppressible stop codon between the display entity and a bacteriophage protein (or fragment thereof), the vector nucleic acid can be transferred into a bacterial cell that cannot suppress a stop codon. In this case, the Fab is not fused to the gene III protein and is secreted into the periplasm and/or media.

Antibody molecules can also be produced in eukaryotic cells. In one embodiment, the antibodies (e.g., scFv's) are expressed in a yeast cell such as Pichia (see, e.g., Powers et al. (2001) J Immunol Methods. 251:123-35), Hanseula, or Saccharomyces.

In some embodiments, antibody molecules are produced in mammalian cells. Preferred mammalian host cells for expressing the clone antibodies or antigen-binding fragments thereof include Chinese Hamster Ovary (CHO cells) (including dhf r̅ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NS0 myeloma cells and SP2 cells, COS cells, and a cell from a transgenic animal, e.g., a transgenic mammal. For example, the cell is a mammary epithelial cell.

In other embodiments, antibody-like molecules in the form of scFv compositions or scFv-Fc fusions are produced. The scFv-Fc fusions comprise multimeric scFv regions covalently linked to hinge regions that are produced in mammalian cells. Following protein synthesis, assembly and secretion, the scFv-Hinge monomers combine at the hinge region through di-sulfide bonds to form scFv-Fc bivalent multimers.

In addition to the nucleic acid sequences encoding the antibody molecule, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced.

In an exemplary system for recombinant expression of an antibody molecule, a recombinant expression vector encoding both the antibody heavy chain and the antibody light chain is introduced into dhfr CHO cells by calcium phosphate-mediated transfection. Within the recombinant expression vector, the antibody heavy and light chain genes are each operatively linked to the same or different enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells can be cultured to allow for expression of the antibody heavy and light chains and intact antibody is recovered from the culture medium. Standard molecular biology techniques can be used to prepare the recombinant expression vector, transfect the host cells, select for transformants, culture the host cells and recover the antibody molecule from the culture medium. For example, some antibody molecules can be isolated by affinity chromatography with a Protein A or Protein G coupled matrix.

For antibody molecules that include an Fc domain, the antibody production system preferably synthesizes antibodies in which the Fc region is glycosylated. For example, the Fc domain of IgG molecules is glycosylated at asparagine 297 in the CH2 domain. This asparagine is the site for modification with biantennary-type oligosaccharides. It has been demonstrated that this glycosylation is required for effector functions mediated by Fc.gamma. receptors and complement C1q (Burton and Woof (1992) Adv. Immunol. 51:1-84; Jefferis et al. (1998) Immunol. Rev. 163:59-76). In one embodiment, the Fc domain is produced in a mammalian expression system that appropriately glycosylates the residue corresponding to asparagine 297. The Fc domain can also include other eukaryotic post-translational modifications.

Antibody molecules can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody molecule and a signal sequence for secretion. The milk produced by females of such transgenic mammals includes, secreted-therein, the antibody of interest. The antibody molecule can be purified from the milk, or for some applications, used directly. The binding properties of a binding agent may be measured by any method, e.g., one of the following methods: BIACORE™. analysis, Enzyme Linked Immunosorbent Assay (ELISA), x-ray crystallography, sequence analysis and scanning mutagenesis.

A RAGE binding agent, particularly an anti-RAGE antibody or fragment, can have a statistically significant effect in one or more of these assays. Exemplary assays for binding properties include the following.

The binding interaction of a RAGE binding agent and a target (e.g., RAGE) can be analyzed using surface plasmon resonance (SPR). SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface. The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden).

Information from SPR can be used to provide an accurate and quantitative measure of the equilibrium dissociation constant (K_{d}), and kinetic parameters, including Kₒₙ and K_{off}, for the binding of a molecule to a target. Such data can be used to compare different molecules. Information from SPR can also be used to develop structure-activity relationships (SAR). For example, the kinetic and equilibrium binding parameters of different antibody molecule can be evaluated. Variant amino acids at given positions can be identified that correlate with particular binding parameters, e.g., high affinity and slow K_{off}. This information can be combined with structural modeling (e.g., using homology modeling, energy minimization, or structure determination by x-ray crystallography or NMR). As a result, an understanding of the physical interaction between the protein and its target can be formulated and used to guide other design processes. binding agents, e.g. antibody molecules that bind to RAGE (such as those described herein) can be used in vitro, ex vivo, or in vivo. They can be incorporated into a pharmaceutical composition, e.g., by combining the RAGE binding agent with a pharmaceutically acceptable carrier. Such a composition may contain, in addition to the RAGE binding agent and carrier, various diluents, fillers, salts, buffers, stabilizers, solubilizers, and other materials well known in the art. Pharmaceutically acceptable materials are generally a nontoxic material that does not interfere with the effectiveness of the biological activity of an RAGE binding agent. The characteristics of the carrier can depend on the route of administration.

The pharmaceutical composition described herein may also contain other factors, such as, but not limited to, other anti-cytokine antibody molecules or other anti-inflammatory agents as described in more detail below. Such additional factors and/or agents may be included in the pharmaceutical composition to produce a synergistic effect with an RAGE binding agent, e.g., anti-RAGE antibody molecule, described herein. For example, in the treatment of allergic asthma, a pharmaceutical composition described herein may include anti-IL-4 antibody molecules or drugs known to reduce an allergic response.

The pharmaceutical composition described herein may be in the form of a liposome in which an RAGE binding agent, e.g., an anti-RAGE antibody molecule, such as one described herein is combined, in addition to other pharmaceutically acceptable carriers, with amphipathic agents such as lipids that exist in aggregated form as micelles, insoluble monolayers, liquid crystals, or lamellar layers while in aqueous solution. Suitable lipids for liposomal formulation include, without limitation, monoglycerides, diglycerides, sulfatides, lysolecithin, phospholipids, saponin, bile acids, and the like. Exemplary methods for preparing such liposomal formulations include methods described in U.S. Pat. Nos. 4,235,871; 4,501,728; 4,837,028; and 4,737,323.

As used herein, the term “therapeutically effective amount” means the total amount of each active component of the pharmaceutical composition or method that is sufficient to show a meaningful patient benefit, e.g., amelioration of symptoms of, healing of, or increase in rate of healing of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

In practicing the method of treatment or use, a therapeutically effective amount of RAGE binding agent, e.g., an anti-RAGE antibody molecule, e.g., an antibody molecule that binds to RAGE and interferes with the formation of a functional RAGE signaling complex (and, e.g., neutralizes or inhibits one or more RAGE-associated activities), is administered to a subject, e.g., mammal (e.g., a human). An RAGE binding agent, e.g., an anti-RAGE antibody molecule, may be administered in accordance with a method described herein either alone as well as in combination with other therapies such as treatments employing cytokines, lymphokines or other hematopoietic factors, cancer therapeutics, or anti-inflammatory agents. When co-administered with one or more agents, an RAGE binding agent, e.g., an anti-RAGE antibody molecule, may be administered either simultaneously with the second agent, or sequentially. If administered sequentially, a physician can select an appropriate sequence for administering the RAGE binding agent in combination with other agents.

Administration of an RAGE binding agent, e.g., an anti-RAGE antibody molecule, used in the pharmaceutical composition can be carried out in a variety of conventional ways, such as oral ingestion, inhalation, or cutaneous, subcutaneous, or intravenous injection. When a therapeutically effective amount of an RAGE binding agent, e.g., an anti-RAGE antibody molecule, is administered by intravenous, cutaneous or subcutaneous injection, the binding agent can be prepared as a pyrogen-free, parenterally acceptable aqueous solution. The composition of such parenterally acceptable protein solutions can be adapted in view factors such as pH, isotonicity, stability, and the like, e.g., to optimize the composition for physiological conditions, binding agent stability, and so forth. A pharmaceutical composition for intravenous, cutaneous, or subcutaneous injection can contain, e.g., an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition may also contain stabilizers, preservatives, buffers, antioxidants, or other additive.

The amount of an RAGE binding agent, e.g., an anti-RAGE antibody molecule, in the pharmaceutical composition can depend upon the nature and severity of the condition being treated, and on the nature of prior treatments that the patient has undergone. The pharmaceutical composition can be administered to normal patients or patients who do not show symptoms, e.g., in a prophylactic mode. An attending physician may decide the amount of RAGE binding agent, e.g., an anti-RAGE antibody molecule, with which to treat each individual patient. For example, an attending physician can administer low doses of antagonist and observe the patient's response. Larger doses of antagonist may be administered until the optimal therapeutic effect is obtained for the patient, and at that point the dosage is not generally increased further. For example, a pharmaceutical may contain between about 0.1 mg to 50 mg antibody per kg body weight, e.g., between about 0.1 mg and 5 mg or between about 8 mg and 50 mg antibody per kg body weight. In one embodiment in which the antibody is delivered subcutaneously at a frequency of no more than twice per month, e.g., every other week or monthly, the composition includes an amount of about 0.7-3.3, e.g., 1.0-3.0 mg/kg, e.g., about 0.8-1.2, 1.2-2.8, or 2.8-3.3 mg/kg.

The duration of therapy using the pharmaceutical composition may vary, depending on the severity of the disease being treated and the condition and potential idiosyncratic response of each individual patient. In one embodiment, the RAGE binding agent, e.g., an anti-RAGE antibody molecule, can also be administered via the subcutaneous route, e.g., in the range of once a week, once every 24, 48, 96 hours, or not more frequently than such intervals. Exemplary dosages can be in the range of 0.1-20 mg/kg, more preferably 1-10 mg/kg. The agent can be administered, e.g., by intravenous infusion at a rate of less than 20, 10, 5, or 1 mg/min to reach a dose of about 1 to 50 mg/m² or about 5 to 20 mg/m².

In one embodiment, an administration of a RAGE binding agent to the patient includes varying the dosage of the protein, e.g., to reduce or minimize side effects. For example, the subject can be administered a first dosage, e.g., a dosage less than a therapeutically effective amount. In a subsequent interval, e.g., at least 6, 12, 24, or 48 hours later, the patient can be administered a second dosage, e.g., a dosage that is at least 25, 50, 75, or 100% greater than the first dosage. For example, the second and/or a comparable third, fourth and fifth dosage can be at least about 70, 80, 90, or 100% of a therapeutically effective amount.

A composition that includes an RAGE binding agent, e.g., an anti-RAGE antibody molecule, can be formulated for inhalation or other mode of pulmonary delivery. Accordingly, the RAGE binding agent can be administered by inhalation to pulmonary tissue. The term “pulmonary tissue” as used herein refers to any tissue of the respiratory tract and includes both the upper and lower respiratory tract, except where otherwise indicated. An RAGE binding agent, e.g., an anti-RAGE antibody molecule, can be administered in combination with one or more of the existing modalities for treating pulmonary diseases.

In one example the RAGE binding agent is formulated for a nebulizer. In one embodiment, the RAGE binding agent can be stored in a lyophilized form (e.g., at room temperature) and reconstituted in solution prior to inhalation. It is also possible to formulate the RAGE binding agent for inhalation using a medical device, e.g., an inhaler. See, e.g., U.S. Pat. No. 6,102,035 (a powder inhaler) and U.S. Pat. No. 6,012,454 (a dry powder inhaler). The inhaler can include separate compartments for the RAGE binding agent at a pH suitable for storage and another compartment for a neutralizing buffer and a mechanism for combining the RAGE binding agent with a neutralizing buffer immediately prior to atomization. In one embodiment, the inhaler is a metered dose inhaler.

The three common systems used to deliver drugs locally to the pulmonary air passages include dry powder inhalers (DPIs), metered dose inhalers (MDIs) and nebulizers. MDIs, the most popular method of inhalation administration, may be used to deliver medicaments in a solubilized form or as a dispersion. Typically MDIs comprise a Freon or other relatively high vapor pressure propellant that forces aerosolized medication into the respiratory tract upon activation of the device. Unlike MDIs, DPIs generally rely entirely on the inspiratory efforts of the patient to introduce a medicament in a dry powder form to the lungs. Nebulizers form a medicament aerosol to be inhaled by imparting energy to a liquid solution. Direct pulmonary delivery of drugs during liquid ventilation or pulmonary lavage using a fluorochemical medium has also been explored. These and other methods can be used to deliver an RAGE binding agent, e.g., anti-RAGE antibody molecule. In one embodiment, the RAGE binding agent is associated with a polymer, e.g., a polymer that stabilizes or increases half-life of the compound.

For example, for administration by inhalation, an RAGE binding agent, e.g., an anti-RAGE antibody molecule, is delivered in the form of an aerosol spray from pressured container or dispenser that contains a suitable propellant or a nebulizer. The RAGE binding agent may be in the form of a dry particle or as a liquid. Particles that include the RAGE binding agent can be prepared, e.g., by spray drying, by drying an aqueous solution of the RAGE binding agent, e.g., an anti-RAGE antibody molecule, with a charge neutralizing agent and then creating particles from the dried powder or by drying an aqueous solution in an organic modifier and then creating particles from the dried powder.

The RAGE binding agent may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges for use in an inhaler or insufflator may be formulated containing a powder mix of an RAGE binding agent, e.g., an anti-RAGE antibody molecule, and a suitable powder base such as lactose or starch, if the particle is a formulated particle. In addition to the formulated or unformulated compound, other materials such as 100% DPPC or other surfactants can be mixed with the RAGE binding agent to promote the delivery and dispersion of formulated or unformulated compound. Methods of preparing dry particles are described, for example, in WO 02/32406.

A RAGE binding agent, e.g., an anti-RAGE antibody molecule, can be formulated for aerosol delivery, e.g., as dry aerosol particles, such that when administered it can be rapidly absorbed and can produce a rapid local or systemic therapeutic result. Administration can be tailored to provide detectable activity within 2 minutes, 5 minutes, 1 hour, or 3 hours of administration. In some embodiments, the peak activity can be achieved even more quickly, e.g., within one half hour or even within ten minutes. An RAGE binding agent, e.g., an anti-RAGE antibody molecule, can be formulated for longer biological half-life (e.g., by association with a polymer such as PEG) for use as an alternative to other modes of administration, e.g., such that the RAGE binding agent enters circulation from the lung and is distributed to other organs or to a particular target organ.

In one embodiment, the RAGE binding agent, e.g., an anti-RAGE antibody molecule, is delivered in an amount such that at least 5% of the mass of the polypeptide is delivered to the lower respiratory tract or the deep lung. Deep lung has an extremely rich capillary network. The respiratory membrane separating capillary lumen from the alveolar air space is very thin (.Itoreq.0 .mu.m) and extremely permeable. In addition, the liquid layer lining the alveolar surface is rich in lung surfactants. In other embodiments, at least 2%, 3%, 5%, 10%, 20%, 30%, 40%, 50%, 60%, 70%, or 80% of the composition of an RAGE binding agent, e.g., an anti-RAGE antibody molecule, is delivered to the lower respiratory tract or to the deep lung. Delivery to either or both of these tissues results in efficient absorption of the RAGE binding agent and high bioavailability. In one embodiment, the RAGE binding agent is provided in a metered dose using, e.g., an inhaler or nebulizer. For example, the RAGE binding agent is delivered in a dosage unit form of at least about 0.02, 0.1, 0.5, 1, 1.5, 2, 5, 10, 20, 40, or 50 mg/puff or more. The percent bioavailability can be calculated as follows: the percent bioavailability=(AUCₙₒₙ₋ᵢₙᵥₐₛᵢᵥₑ/AUC_{i.v.} or s.c.).times.(dose_{i.v.} or s.c./dose.ₙₒₙ₋ᵢₙᵥₐₛᵢᵥₑ)×100.

Although not necessary, delivery enhancers such as surfactants can be used to further enhance pulmonary delivery. A “surfactant” as used herein refers to a RAGE binding agent having a hydrophilic and lipophilic moiety, which promotes absorption of a drug by interacting with an interface between two immiscible phases. Surfactants are useful in the dry particles for several reasons, e.g., reduction of particle agglomeration, reduction of macrophage phagocytosis, etc. When coupled with lung surfactant, a more efficient absorption of the RAGE binding agent can be achieved because surfactants, such as DPPC, will greatly facilitate diffusion of the compound. Surfactants are well known in the art and include but are not limited to phosphoglycerides, e.g., phosphatidylcholines, L-alpha-phosphatidylcholine dipalmitoyl (DPPC) and diphosphatidyl glycerol (DPPG); hexadecanol; fatty acids; polyethylene glycol (PEG); polyoxyethylene-9-; auryl ether; palmitic acid; oleic acid; sorbitan trioleate (Span 85); glycocholate; surfactin; poloxomer; sorbitan fatty acid ester; sorbitan trioleate; tyloxapol; and phospholipids.

In one embodiment, a RAGE binding agent, e.g., an anti-RAGE antibody molecule or RAGE specific antigen binding polypeptide, is physically associated with a moiety that improves its stabilization and/or retention in circulation, e.g., in blood, serum, lymph, bronchopulmonary lavage, or other tissues, e.g., by at least 1.5, 2, 5, 10, or 50 fold.

For example, an RAGE binding agent, e.g., an anti-RAGE antibody molecule, can be associated with a polymer, e.g., a substantially non-antigenic polymers, such as polyalkylene oxides or polyethylene oxides. Suitable polymers will vary substantially by weight. Polymers having molecular number average weights ranging from about 200 to about 35,000 (or about 1,000 to about 15,000, and 2,000 to about 12,500) can be used.

For example, a RAGE binding agent, e.g., an anti-RAGE antibody molecule, can be conjugated to a water soluble polymer, e.g., hydrophilic polyvinyl polymers, e.g. polyvinylalcohol and polyvinylpyrrolidone. A non-limiting list of such polymers includes polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; branched or unbranched polysaccharides which comprise the saccharide monomers D-mannose, D- and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextran sulfate, dextran, dextrins, glycogen, or the polysaccharide subunit of acid mucopolysaccharides, e.g. hyaluronic acid; polymers of sugar alcohols such as polysorbitol and polymannitol; heparin or heparan.

The conjugates of an RAGE binding agent, e.g., an anti-RAGE antibody molecule, and a polymer can be separated from the unreacted starting materials, e.g., by gel filtration or ion exchange chromatography, e.g., HPLC. Heterologous species of the conjugates are purified from one another in the same fashion. Resolution of different species (e.g. containing one or two PEG residues) is also possible due to the difference in the ionic properties of the unreacted amino acids. See, e.g., WO 96/34015.

Use of Rage Binding Agents to Modulate One or More RAGE-Associated Activities In Vivo

In yet another aspect, the invention features a method for modulating (e.g., decreasing, neutralizing and/or inhibiting) one or more associated activities of RAGE in vivo by administering an RAGE binding agent, e.g., an anti-RAGE antibody molecule, described herein in an amount sufficient to inhibit its activity. An RAGE binding agent can also be administered to subjects for whom inhibition of an RAGE-mediated inflammatory response is required. These conditions include, e.g., airway inflammation, asthma, fibrosis, eosinophilia and increased mucus production.

The efficacy of an RAGE binding agent, e.g., an anti-RAGE antibody molecule, described herein can be evaluated, e.g., by evaluating ability of the antagonist to modulate airway inflammation in cynomolgous monkeys exposed to an Ascaris suum allergen. An RAGE binding agent, particularly one that inhibits at least one RAGE activity, can be used to neutralize or inhibit one or more RAGE-associated activities, e.g., to reduce RAGE mediated inflammation in vivo, e.g., for treating or preventing RAGE-associated pathologies, including asthma and/or its associated symptoms.

In one embodiment, an RAGE binding agent, e.g., an anti-RAGE antibody molecule, e.g., pharmaceutical compositions thereof, is administered in combination therapy, i.e., combined with other agents, e.g., therapeutic agents, that are useful for treating pathological conditions or disorders, such as allergic and inflammatory disorders. The term “in combination” in this context means that the agents are given substantially contemporaneously, either simultaneously or sequentially. If given sequentially, at the onset of administration of the second compound, the first of the two compounds is preferably still detectable at effective concentrations at the site of treatment.

For example, the combination therapy can include one or more RAGE binding agents, e.g., anti-RAGE antibodies and fragments thereof, e.g., that bind to RAGE and interfere with the formation of a functional RAGE signaling complex, co-formulated with, and/or co-administered with, one or more additional therapeutic agents, e.g., one or more cytokine and growth factor inhibitors, immunosuppressants, anti-inflammatory agents, metabolic inhibitors, enzyme inhibitors, and/or cytotoxic or cytostatic agents, as described in more detail below. Furthermore, one or more an RAGE binding agent, e.g., an anti-RAGE antibody molecule, may be used in combination with two or more of the therapeutic agents described herein. Such combination therapies may advantageously utilize lower dosages of the administered therapeutic agents, thus avoiding possible toxicities or complications associated with the various monotherapies.

Detection of Antibodies, scFv and scFv Fusions

Antibodies and scFv fragments and fusions are typically detected by binding of labeled antibodies that react specifically with a portion of human Fc region. In addition, efficiency of binding of antibodies and fragments and fusions thereof can be detected indirectly through competition assays with antibodies known to bind the same antigen and/or same or overlapping epitope. ScFvs were selected for improved binding by competition assays with parental humanized M4 (XT-M4) or with the original M4 rat monoclonal antibody to RAGE.

To determine whether RAGE antibodies or fragments or antigen-binding polypeptides affect the binding of a RAGE ligand (HMGB1; Sigma, St. Louis, Mo.) to RAGE, competition ELISA binding assays are performed. Ninety-six well plates are coated with 1 μg/ml of HMGB1 overnight at 4° C. Wells are washed and blocked and exposed to 100 μl of pre-incubated mixtures of RAGE-Fc or TrkB-Fc (a non-specific Fc control), at 0.1 μ.g/ml, plus various forms of the indicated antibody preparation (dilutions of immune sera, hybridoma supernatants or purified antibodies or fragments) for 1 hour at room temperature. Plates are washed with PBS, pH 7.2 and ligand-bound recombinant human RAGE-Fc is detected with the use of peroxidase-conjugated goat, anti-human IgG (Fc.gamma.) (Pierce, Rockford, Ill.), followed by incubation with the substrate TMB (BioFX Laboratories Owings Mills, Md. Laboratories Owings Mills, Md.). Binding of recombinant human RAGE-Fc to ligand without any antibodies, polypeptides or fragments or with diluted pre-immune serum was used as a control and defined as 100% binding.

The binding of selected anti-RAGE polypeptides to human and murine RAGE and to the V domains of human and murine RAGE is analyzed by BIACORE™ direct binding assay. Assays are performed using human or murine RAGE-Fc coated on a CM5 chip at high density (2000 RU) using standard amine coupling. Solution of the anti-RAGE antibodies at two concentrations, 50 and 100 nm, are run over the immobilized RAGE-Fc proteins in duplicate. BIACORE™ technology utilizes changes in the refractive index at the surface layer upon binding of the anti-RAGE antibodies to the immobilized RAGE antigen. Binding is detected by surface plasmon resonance (SPR) of laser light refracting from the surface.

The binding of chimeric antibody XT-M4, humanized XT-M4 antibodies and scFv-Fc fusions to soluble human RAGE (hRAGE-SA) and soluble murine RAGE (mRAGE-SA) was measured by BIACORE™ capture binding assay, HTRF or ELSA analytical methods. The BIACORE™ assays are performed by coating anti-human Fc antibodies onto a CM5 BIA chip with 5000 RU (pH 5.0, 7 min.) in flow cells 1-4. Each antibody is captured by flowing at 2.0 μg/ml over the anti-Fc antibodies in flow cells 2-4 (flow cell 1 is used as a reference). Solutions of a purified soluble human streptavidin-tagged RAGE (hRAGE-SA) at concentrations of 100 nM, 50 nM, 25 nM, 12.5 nM, 6.25 nM, 3.125 nM, 1.25 nM and 0 nM are flowed over the immobilized antibodies in duplicate, with dissociation for 5 minutes, and kinetic rate constants (k.ₐ and k_{d}) and association and dissociation constants (Kₐ and K_{d}) for binding to hRAGE-SA are determined. Biotin labeled antigen is more compatible with affinity driven selections in solution. Selections are performed either in an equilibrium mode at a decreasing antigen concentration relative to starting affinity or in a kinetic mode where improved off rate is specifically selected for using competition with unlabelled antigen over an empirically determined time frame. Standard procedures for phage display are used.

The M4 antibody was humanized to decrease immunogenicity and improve the pharmacokinetic and safety profiles of the molecule. Humanization was carried out against the DP-54/DPK9 germline frameworks. After a series of back-mutations and testing, a final humanized antibody was derived which had only three amino acid changes in the V_{L} (Y36F, L46R, K45R, and G66R). The HTRF competition assay with labeled parental M4 antibody was used to rank potency of selected variants and to maintain the M4 epitope. Variations of humanized anti-RAGE M4 (XT-M4) have been described in U.S. Patent Publication 2007/0286858.

### EXAMPLES

### Example 1

### Reformatting and Mutagenesis of Parental Humanized XT-M4 as an ScFv Antibody

Prior to mutagenesis and testing for improved potency the parental antibody XT-M4 was reformatted as an scFv in both the V_{L}-V_{H} format (FIG. 1 and SEQ ID NO:75) and the V_{H}-V_{L} format, incorporating a flexible linker sequence [DGGGSGGGGSGGGGSS; SEQ ID NO:41]. Both formats were functional, but the V_{L}-V_{H} format was chosen for optimization. Restriction sites were also incorporated at either end of XT-M4 scFv to facilitate convenient reformatting to scFv-Fc fusion proteins utilizing general recombinant DNA techniques. Assembled scFv antibody fragments were synthesized from overlapping oligonucleotides, digested with Sfi1 restriction enzyme and cloned into the phage display vector pWRIL-1.

Parental antibody in the scFv format was mutagenized and screened for improved potency. Mutagenesis was performed using standard techniques, including oligonucleotide site-specific and error-prone PCR mutagenesis. Libraries of mutant clones were selected for increased antigen binding utilizing either phage display or Ribosome Display technology.

Antibody VH is numbered sequentially from residue 124 to 242 of SEQ ID NO:75 (See also SEQ ID NO:2). Antibody VL is numbered sequentially from residue 1 to 107 of SEQ ID NO:75 (See also SEQ ID NO:1). The CDR's of the VL include and are defined by amino acid numbers 24 to 34 of FIG. 1 and SEQ ID NO: 75 (CDRL-1), amino acid numbers 50 to 56 of FIG. 1 and SEQ ID NO: 75 CDRL-2) and amino acid numbers 89 to 97 The CDR's of the VH include and are defined by amino acid numbers 149 to 158 of FIG. 1 and SEQ ID NO: 75 (CDRH-1), amino acid numbers 173 to 189 of FIG. 1 and SEQ ID NO: 75 (CDRH-2) and amino acid numbers 222 to 231 of FIG. 1 and SEQ ID NO: 75 (CDRH-3).

A Ribosome Display library of variants was created by error prone PCR. This allowed the introduction of diversity over the whole length of the molecule and allowed the isolation of potentially beneficial mutations in CDRs other than VH-CDR3, framework residues and vernier regions. This approach is analogous to the natural process of somatic hypermutation. An added feature of this approach is the potential mapping of the functional antibody paratope, the definition of mutational ‘Hotspots’ and potential isolation of mutations that enhance VH/VL domain interactions. Due to the huge molecular diversity that can be generated by error prone PCR, this approach was only used in conjunction with ribosome display. The XT-M4-error prone PCR product was cloned into an appropriate ribosome display vector and had an estimated size of 5×10¹² Two phage display libraries were constructed targeting diversity into either the VH-CDR3 loop or the VL-CDR3 loop. The VH-CDR3 was aggressively mutated using total randomization using sequential NNS mutagenic codons spanning the length of the VH-CDR3 in blocks of two stretches of 6 codons overlapping by two codons to cover the VH-CDR3 of length 10 codons. The VL-CDR3 was subjected to a lower mutational load and a codon based strategy was taken. This approach aimed to mimic the natural amino acid diversity at each position within this loop using collated sequence alignment of natural V genes in the public database. The V_{H}-CDR3 randomised library had a size of 1.2×10⁹ and the V_{L}-CDR3 based library was 5×10⁸. The frequency and distribution of mutations in both CDR3 libraries (determined by sequencing), was consistent with the theoretical diversity introduced by the oligonucleotide design.

### Example 2

### Selection of scFv Clones with Improved Affinity for Human and Mouse RAGE

Increases in binding to RAGE antigen were detected with the aid of a competition assay using parental XT-M4 antibody. Both phage display and ribosome display libraries were selected for affinity improved variants by incubating with biotin labeled hRAGE-Fc, recovering binding clones using streptavidin magnetic beads and washing away non binding variants. Sequential rounds of selection were carried on decreasing antigen concentrations to drive the preferential recovery of higher affinity variants. Clones recovered after selection were subsequently screening for improved binding to hRAGE-Fc using HTRF. This is an assay that measures the decrease in fluorescence upon binding of parental europium cryptate-labeled XT-M4 to RAGE in the presence of competing test scFv antibodies. In these assays, periplasmic preparations of scFv were prepared from bacterial cultures and added in increasing concentrations to a combination of parental antibody and antigen. The ability of the scFV to compete with parental XT-M4 antibody to bind to Biotin-labeled RAGE-Fc was determined. In the presence of avidin-XL665 complex labeled XT-M4 bound to biotinylated RAGE-FC was detected by Fluorescence. Increasing amounts of scFv that competed with the XT-M4 for binding to biotinylated-RAGE-Fc was detected as a decrease in fluorescence. A sequential process of screening was used to focus in on a smaller number of clones with the greatest competition in HTRF assay

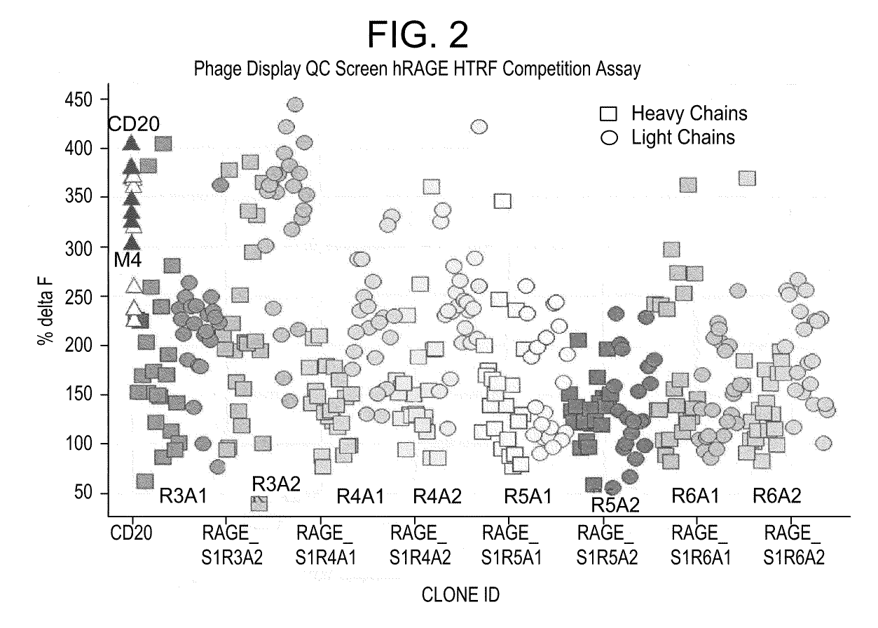

High-throughput HTRF analysis of individual rounds of selections on human RAGE for Phage Display clones is shown in FIG. 2. Empty triangles represent parental XT-M4 scFv. Filled triangles represent negative control anti CD20 scFv. Circles represent clones derived from the VL-CDR3 library and squares represent clones derived from the VH-CDR3 libraries. All analyses were carried out as single point assays using unpurified periplasmic preparations of scFv proteins. Clones towards the top of the figure are negative non-binding clones and as selections proceed from left to right the number of non binding clones decreases.

High-throughput HTRF analysis of individual rounds of selections on human RAGE are shown for ribosomal display clones in FIG. 3. Range of negative control (CD20 ScFv, parental wild-type (XTM4 scFv) and positive control (H8 ScFv) are indicated on the y axis which measures the change in fluorescence in competition HTRF. The clones with improved binding are boxed.

An increase in the number of highly competing clones retrieved was observed after each round of selection, when compared to parental XT-M4 scFv.

Selected V_{L}-CDR3 variants were found to be less potent than the V_{H}-CDR3 variants, suggesting a greater importance of the V_{H}-CDR3 in determining antigen binding. It was also observed that the GGDI motif at the 5′ end of the V_{H}-CDR3 sequence was not tolerant of mutation (an observation further confirmed using ribosome display strategy). A particular mutation in the heavy chain CDR3, F106L, was identified in the V_{H}-CDR3 that was present in the vast majority of selected variants. F1061 was also observed in several clones, but this mutation was not associated with the same affinity gains observed for F106L. Sequence analysis of improved variants showed that there were several distinct families of clones.

A large family of closely related improved clones was found to have a “charge-hydrophobe-small” motif in the center of the loop (positions 103, 104, 105), predominantly comprised of K/R-V-G/S sequences. A second family of improved clones had a different motif at positions 103-105; comprised of ‘hydrophobe-charge-small’ (UV-D-S/G), or ‘hydrophobe-hydrophobe-small’ (L-V-G/S) sequences. In almost all clones sequenced there was a preference for a small amino acid (S, G, occasionally M) at position 105. This represents maintenance of the wild-type amino acid chemistry at this position. The T103K/R/L and T104V/D mutations represent significant changes in chemistry at these positions. The vast majority of improved clones exhibited a preference for a charged residue (D, R, H) to the c-terminal side of F106L (position 107), with the natural amino acid at this position (D) being predominantly preferred. However, the highest overall affinity clone identified (clone 3G5) carries a proline in this position. The last position in the CDR3 (Y108) was generally variable amongst the total population, but was mostly maintained as one of the large aromatic residues most often found at this position in natural antibodies (Y, F). Gains in affinity were somewhat less successful in the VL than the VH. Tables 1 and 2 below represent Phage Display (Table 1) and Ribosome Display (Table 2) clones that were selected for increased affinity for RAGE binding. Clones highlighted with an “*” in Table 1 and all clones except S2R4A4_{—}6G2 in Table 2 were reformatted as scFv-Fc fusions, as discussed below in greater detail and mutations are shown in table 12.

TABLE 1
IC50 VALUES FOR PHAGE DISPLAY CLONES.
“X” REPRESENTS ANY AMINO ACID

| Clone ID | LCDR3 | HCDR3 | IC50 (nM) |
|---|---|---|---|
| M4WT | LEFDEHPLT | GGDITTGFDY | 33.2 |
| 3A10 | LEFSLSRS | | Nd |
| 3B7 | LQFDSHPLT | | 7.35 |
| 3B9 | LQFDNHPLT | | 10.59 |
| 3C7 | LQYDAHPXT | | 9.23 |
| 3E8 | LQFDVHPLT | | 7.21 |
| 3G8 | LQYDAHPLT | | 8.08 |
| 6A8 | XXFXXHPLT | | 3.14 |
| 6A10 | LQFDAHPLT | | 6.94 |
| 6A11 | LQFDSHPLT | | 32.75 |
| 6A12 | LQYDAHPLT | | 8.27 |
| 6B8 | XXFDXHPLT | | 3.34 |
| 6C1 | LELDEHPLT | | 4.44 |
| 6C10 | LQFDEHPLT | | 7.38 |
| 6C11 | LQYDAHPLT | | 9.03 |
| 3A6* | | GGDILVSLDV | 0.474 |
| 3B2* | | GGDILVGLDY | 0.917 |
| 3B4* | | GGDIREGLRY | 1.306 |
| 3C6 | | GGDIVVGLDH | 1.259 |
| 3D2* | | GGDIRVSLDH | 1.274 |
| 3D5 | | GGDIKVGLDL | 2.998 |
| 3D6 | | GGDIRVMLDL | 2.924 |
| 3G3 | | GGDIKVSLDH | 1.44 |
| 3G5* | | GGDILDSLPY | 0.87 |
| 6B2* | | GGDIRVGLDV | 1.774 |
| 6B6* | | GGDIIISLDW | 0.645 |
| 6C1 | | GGDIKVGLDN | 4.44 |
| 6C2* | | GGDIKVSLDR | 0.731 |
| 6C3* | | GGDITLGLDV | 1.527 |
| 6D5 | | DDDIKVSLDQ | 1.458 |
| 6E3 | | GGDIRVSLDF | 1.747 |
| 6E5 | | GGDIRVMLDV | 2.25 |
| 6G4* | | GGDILDSLHF | 2.03 |

TABLE 2
IC50 VALUES FOR RIBOSOME DISPLAY CLONES

| Clone | IC50 (nM) |
|---|---|
| XT-M4 | 33.0 |
| S2R4A4_6G2 | 1.3 |
| S2R4A3_10H6 | 0.3 |
| S2R3A1_8G9 | ~2.4 |
| S2R4A3_10D8 | 2.6 |
| S1R5A1_3B3 | 2.2 |
| S2R4A4_1G6 | 1.2 |
| S2R3A1_2E6 | 1.9 |
| S2R4A3_5A3 | ~3.0 |
| S1R5A1_10G10 | 16.3 |
| S2R3A1_5H3 | 8.2 |

Sequence analysis of 261 Ribosome Display clones from sequential rounds of selection with functional binding to coated hRAGE-Fc in ELISA showed a diverse spread of mutations in both the V_{H} and V_{L} domain distributed across both CDR's and the framework regions. Furthermore, residues were defined that do not tolerate mutations. Some evidence of dominant mutations that were carried through consecutive rounds of selection was also identified, indicating selective pressure for certain clones.

Amino Acid Substitutions

Tables 3 and 4 below show a list of the residue positions, which correlated with improved anti-RAGE potency in Ribosome Display clones. This list is derived from a panel of 123 clones, which had improved potency in a single point HTRF assay.

TABLE 3
MUTATIONAL SPECTRUM AT KEY RESIDUES IN
VL DOMAIN CORRELATED WITH IMPROVED POTENCY
IN RIBOSOME DISPLAY DERIVED CLONES

| Selected mutation in panel | Other mutations selected at |
|---|---|
| of 9 best clones | that position and frequency |
| R18G | G(1), I(2), K(1), S(1) |
| R24Q | L(1), Q(1) |
| I31F | F(9), H(1), N(2), S(1), T(1), V(2) |
| Y49D | D(2) |
| G68S | S(1) |
| K103R | E(2), N(2), R(2) |

TABLE 4
MUTATIONAL SPECTRUM AT KEY RESIDUES IN
VH DOMAIN CORRELATED WITH IMPROVED POTENCY
IN RIBOSOME DISPLAY DERIVED CLONES

| Selected mutation in panel | Other mutations selected at |
|---|---|
| of 9 best clones | that position and frequency |
| Q3L | H(2), R(1) |
| G9V | D(2), V(1) |
| G10E | E(1) |
| G16S | S(2) |
| T35S | S(2) |
| D56E | A, E, G, N, Y(1 each) |
| Q82K | K(1), R(1) |
| A97T | S(2), T(3) |
| T104N | N(3), P(1) |
| F106L | I(7), L(6) |
| V117E | E(1), L(1), M(2) |

Selection of Invariant Residues.

In analysis of 261 clones with functional binding to human RAGE (hRAGE) in ELISA and 123 clones that had improved potency in competition HTRF assay residues that were invariant and could not tolerate mutation could be defined. These residues are predicted to have either a direct or indirect role in binding RAGE and provide an insight into the antibody paratope. Table 5 below shows the mutational spectrum in functional clones isolated from round 3 and round 4 selections of the ribosome display library.

TABLE 5

| V region | V_{L} domain | V_{H} domain |
|---|---|---|
| FW1 | Q6, P8, S10, | S7, A24, |
| CDR1 | A25, S26, G30 | — |
| FW2 | P44, I48 | V37, Q39, |
| CDR2 | | Y59 |
| FW3 | R61, S65, E81, C88 | — |
| CDR3 | E90, P95 | G99, G100, D101, |
| | | I102 |
| FW4 | G99 | — |

TABLE 6

| Parental residue in | Mutations selected at that position |
|---|---|
| VH-CDR3 loop | correlated with improved potency |
| G 99 | Invariant |
| G 100 | Invariant |
| D 101 | Invariant |
| I 102 | Invariant |
| T 103 | L, R, I, K |
| T 104 | V, E, D, I, N, L |
| G 105 | S |
| F 106 | L, I |
| D 107 | R, P, H |
| Y 108 | H, V, W, R, F |

Mutations in scFv Flexible Linker

Mutations were selected with improved potency that contained mutations in the flexible linker between the light and heavy chain regions. Within this population several linker mutations with a distinct bias towards hydrophilic amino acids were identified.

The following starting linker sequence of amino acids was used to generate scFVs having a VL fused to a VH domain:

DGGGSGGGGSGGGGSS-SEQ ID NO:41 Positions that were substituted in clones showing improved binding included the following: D>N at position 1; G>D at position 6; G>N position 7. G>E at position 9, S>F at position 10; G>AR or E at position 12, G>R at position 13 and S>G or N at position 15 of SEQ ID NO:41. These substitutions in the flexible linker are also described in SEQ ID NO:49.

Mutations in VH-CDR3

Substitutions of amino acids in V_{H} CDRH-3 that correlated with improved potency in HTRF assay are summarized in Table 6. The parental residues and mutations selected at each position in VH-CDR3 are shown derived by both phage display and ribosome display. V_{H} CDR3 spans from amino acid residues G99 to Y108 and invariant refers to residues that do not tolerate mutation.

Selection of Additional Amino Acid Substitutions in Framework and CDR Regions.

Tables 7 and 8 show the mutational spectrum of high affinity clones from a panel of 123 Ribosome Display clones that had improved potency in a single point HTRF assay.

TABLE 7
LIGHT CHAIN CLONES

| Selected mutation in panel | Other mutations selected at |
|---|---|
| of 9 bestV_{L} clones | that position and frequency |
| R18G | G(1), I(2), K(1), S(1) |
| R24Q | L(1), Q(1) |
| I31F | F(9), H(1), N(2), S(1), T(1), V(2) |
| Y49D | D(2) |
| G68S | S(1) |
| K103R | E(2), N(2), R(2) |

TABLE 8
HEAVY CHAIN CLONES

| Selected mutation in panel | Other mutations selected at |
|---|---|
| of 9 best V_{H} clones | that position and frequency |
| Q3L | H(2), R(1) |
| G9V | D(2), V(1) |
| G10E | E(1) |
| G16S | S(2) |
| T35S | S(2) |
| D56E | A, E, G, N, Y(1 each) |
| Q82K | K(1), R(1) |
| A97T | S(2), T(3) |
| T104N | N(3), P(1) |
| F106L | I(7), L(6) |
| V117E | E(1), L(1), M(2) |

### Example 3

### Replacement of Invariant Residues with Equivalent Amino Acids

scFv mutagenesis is performed as described above by directed amino acid substitutions in Framework and CDR residues that have already been shown to tolerate substitutions. Additional substitutions with proposed rationale are shown below in Tables 9 and 10.

TABLE 9
LIGHT CHAIN CLONES

| Selected mutation in panel | |
|---|---|
| of 9 best V_{L} clones | Correlated biophysical property |
| R18G | |
| R24Q | |
| I31F | Y, W, H as bulky aromatic amino acids. |
| Y49D | |
| G68S | |
| K103R | Polar amino acids. Other potential |
| | candidates are Q, K, D, H, W, Y |

TABLE 10
HEAVY CHAIN CLONES

| Selected mutation in panel | |
|---|---|
| of 9 best V_{H} clones | Correlated biophysical property |
| Q3L | Large amino acids- K, W, Y |
| G9V | |
| G10E | |
| G16S | |
| T35S | |
| D56E | |
| Q82K | Large polar amino acids- H, W, Y |
| A97T | |
| T104N | |
| F106L | Large Non-polar amino acids M and V |
| V117E | |

### Example 4

### Reformatting of scFv Clones to scFv-Fc Fusion Proteins

Based on IC50 values in the HTRF assay described above, the 10 best clones from phage display (Table 1) and ribosome display (Table 2) selections were chosen for scFv-Fc reformatting. In the case of the ribosome display clones, other secondary criteria were also considered in order to make the top 10 selection (i.e. clones had to have a mutation with an amino acid frequency of >4 in the population of 123 sequences, clones carrying frequently occurring mutations and clones considered to be carrying mutations potentially positioned at the V_{L}/V_{H} interface).

The initial design of the parental XT-M4 scFv construct incorporated BssHII and Bcl1 restriction sites at the 5′ and 3′ end of the scFv sequence, respectively to facilitate direct reformatting into Fc fusions using the chosen acceptor vector.

The acceptor vector contained a wildtype (wt) IgG constant region (Fc), with a eukaryotic promoter and eukaryotic and bacterial origins of replication for transfer and expression in bacteria and eukaryotic organisms. It also contained a multiple cloning site for integration of one or more variable region binding domains and allows for expression of the variable region(s) as part of an FV-Fc fusion protein. Nucleic acids encoding selected scFv were cloned into pre-the pSMED vector operably linked and fused at the protein level with an Fc constant region. The recombinant plasmid contained an open reading frame comprising the scFv coding region amino to the Fc region containing protein coding sequences for the hinge region followed by the CH1 and CH2 regions of a human IgG.

The recombinant plasmid described above was transfected into COS cells and the scFv-Fc fusions constructs were expressed. Following expression in COS cells, the scFv utilizes the dual hinge regions to form a bivalent scFv-Fc fusion construct The panel of selected clones derived by phage display (n=10) and ribosome display (n=10) were converted to Fc fusions as described above for the parent XT-M4 [Note: one of the ribosome display clones was lost due to the generation of a internal Bcl1 site by random mutagenesis.

These were expressed transiently in COS cells and purified by Protein A affinity chromatography, followed by buffer exchange into PBS. SDS-PAGE analysis of the purified proteins indicated that the level of purity was high and did not detect any obvious aggregation or degradation products. SEC (size exclusion chromatography) analysis of each of the clones was also carried out to detect formation of high molecular weight aggregates (HMW). Overall, the level of HMW formation was low for both phage display and ribosome display clones. The ribosome display clones discovered herein in particular have a very favorable SEC profile with low levels of aggregation. Without being bound by theory, low level aggregation may be due to the fact that these scFv molecules have been subjected to random error-prone PCR across the whole length of the sequence and in this sense have evolved as a single unit. Clones 10H6, 10D8 and 2E6 carried a mutation in the flexible linker to Asn residue (D108N, G114N and S122N respectively of SEQ ID NO:75 and FIG. 1) which could also be correlated with improved biochemical characteristics. For SEC analysis of scFv-Fc fusion proteins. All samples were run at a concentration of 60 μg/ml in 50 mM sodium phosphate buffer, pH 7.5.

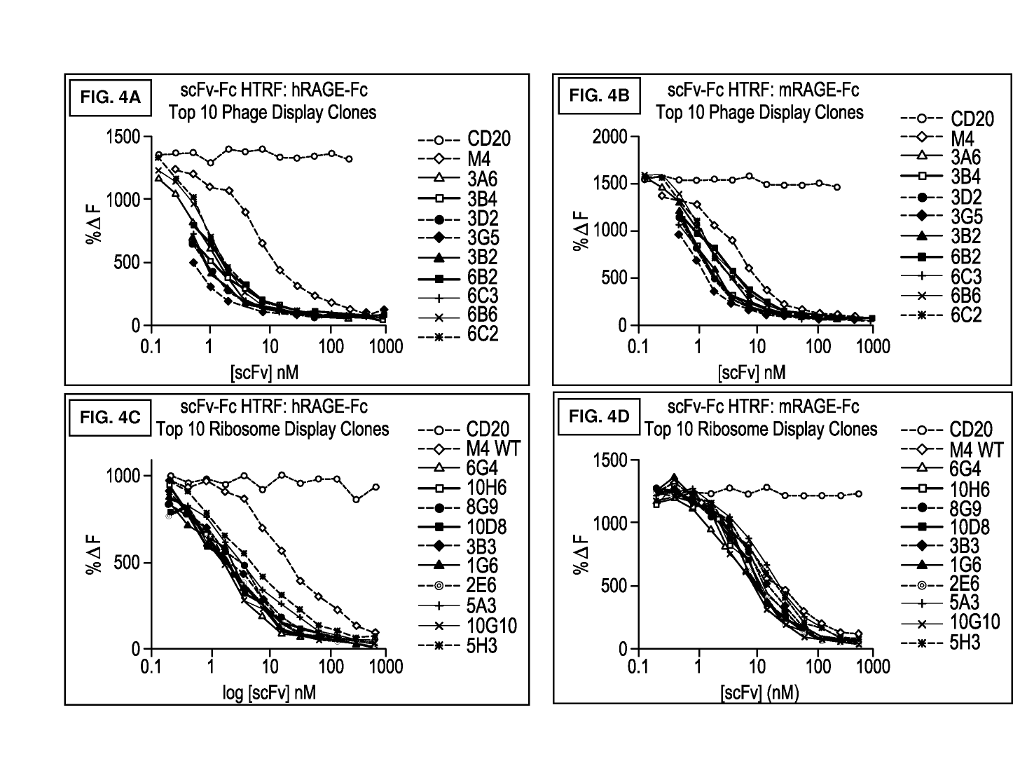

Purified scFv-Fc proteins were also subjected to HTRF titration as above and this confirmed affinity improvements in a bivalent format. In most cases further improvements were seen in going from scFv to a bivalent fusion. Both phage and ribosome display clones showing improved potency were reformatted to scFv-Fc fusions. The HTRF titration analysis was carried out for both human and murine RAGE as shown in FIGS. 4 and 5.

### Example 5

### Characterization of scFv-Fc Fusion Proteins

BIAcore analysis and kinetic constant calculation used RAGE-SA directly immobilized on a CM5 BIACORE surface with scFv-Fc proteins injected over the surface for 3 min with a dissociation period of 5 min. In summary, mutant clones were significantly improved with improvements in kd values ranging from 7 to 69 fold for the phage display clones and 4 to 67 fold for the ribosome display clones. These results are shown as kd values are listed in Table 12 below.

### Example 6

### Binding of scFv-Fc Proteins to CHO-Rage Cells

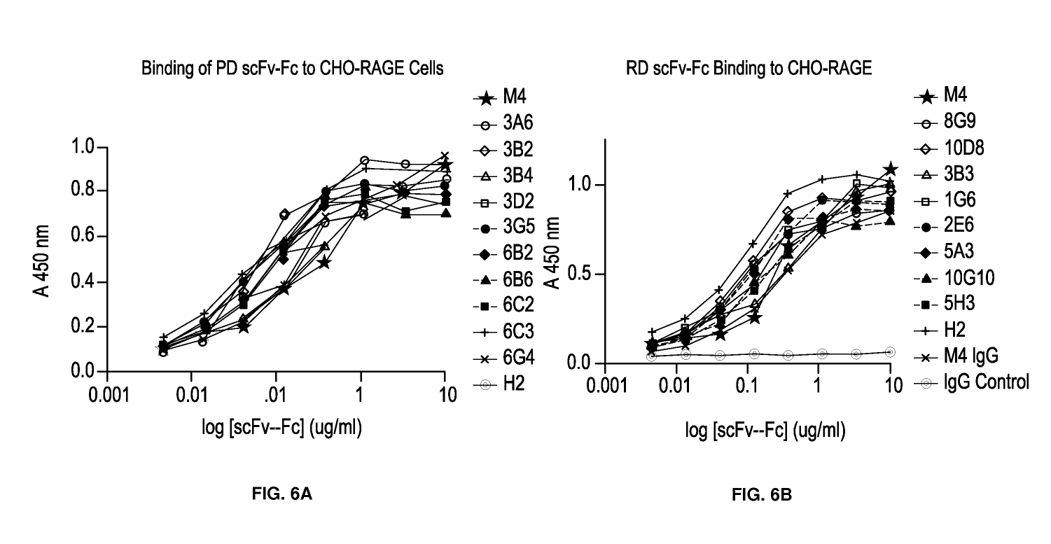

Binding of scFv-Fc proteins to CHO-RAGE cells was conducted to ensure that selected clones also exhibited improved binding to authentic cell surface expressed RAGE target. Improved EC50 values were observed of between 5-14 fold over parental XT-M4 scFv-Fc fusions. Stably transfected Chinese Hamster Ovary (CHO) cells were engineered to express murine and human RAGE full length proteins. The murine and human RAGE cDNAs were cloned into the mammalian expression vector pSMED, linearized and transfected into CHO cells using lipofectin methods (Kaufman, R. J., 1990, Methods in Enzymology 185:537-66; Kaufman, R. J., 1990, Methods in Enzymology 185:487-511; Pittman, D. D. et al., 1993, Methods in Enzymology 222: 236). Cells were further selected in 20 nM methotrexate and cell extracts were harvested from individual clones and analyzed by SDS sodium dodecyl sulfate-polyacrylamide gel electrophoresis (SDS-PAGE) and Western blotting to confirm expression. These results are shown below in Table 11 and also in FIG. 6. Absorbance 450 nm values were corrected for the same clones binding to control CHO cells not expressing RAGE. EC50 values were calculated after curve fitting using GraphPad prism and are expressed in μg/ml. EC50 is the effective concentration that gives 50% of maximum value in ug/ml of scFv-Fc protein.

TABLE 11

| Clone ID Phage | | Clone ID Ribosome | |
|---|---|---|---|
| Display | EC50 | display | EC50 |
| H2 IgG | 0.068 | H2 IgG | 0.0897 |
| M4 IgG | 0.312 | M4 IgG | 0.266 |
| M4 scFv-Fc | 0.408 | M4 scFv-Fc | 0.388 |
| 3A6 | 0.0673 | 8G9 | 0.077 |
| 3B2 | 0.0335 | 10D8 | 0.092 |
| 3D2 | 0.1363 | 1G6 | 0.1875 |
| 3G5 | 0.0565 | 2E6 | 0.1134 |
| 6B2 | 0.0816 | 5A3 | 0.096 |
| 6C2 | 0.0285 | 10G10 | 0.1334 |
| 6C3 | 0.155 | IgG control | N/A |
| 6B6 | 0.0285 | 3B3 | 0.541 |
| 6G4 | 0.1721 | | |

Based on the foregoing examples, it was concluded that in general, potency was improved after reformatting scFv to scFv-Fc fusion. Furthermore, dominant mutations were identified using different mutagenesis strategies, with V_{H}-CDR3 F106L mutation being derived independently using different approaches. This residue is clearly a key determinant of potency improvement.

Affinity gains using phage display and aggressive V_{H}-CDR3 targeted mutagenesis were generally higher compared to ribosome display in conjunction with a random mutagenesis strategy. Ribosome display derived clones tended to have improved biochemical properties with less dimerization and aggregation compared to phage display derived clones.

### Table 12

This summary table shows the results of biological activities for the ribosome display and phage display scFv-Fc antibody fusions that displayed the most improved binding characteristics. Selected Phage display and Ribosome display clones with improved binding to Rage were characterized with respect to the identified characteristics.

TABLE 12
Summary data of optimised antibodies derived from XTM4 (VH 2.0, VL 2.11)

| | | | | scFv | Fold gain scFv | ScFv-Fc |
|---|---|---|---|---|---|---|
| Clone ID | Technology | Mutations VL | Mutations VH | Hu-IC50 | (hRAGE) | Hu-IC50 |
| CD20 | control | N/A | N/A | N/A | N/A | N/A |
| M4 | parental | — | GGDITTGFDY # | 33 | N/A | 7.52 |
| 3A6 | PD | — | GGDILVSLDV | 0.47 | 70 | 0.76 |
| 3B2 | PD | — | GGDILVGLDY | 0.92 | 35.8 | 0.22 |
| 3B4 | PD | — | GGDIRFGLDY | 1.31 | 25.1 | 0.3 |
| 3D2 | PD | — | GGDIRVSLDH | 1.27 | 26 | 0.24 |
| 3G5 | PD | — | GGDILDSLFY | 0.87 | 37.9 | 0.12 |
| 6B2 | PD | — | GGDIRYGLDV | 1.77 | 18.6 | 1.22 |
| 6B6 | PD | — | GGDIDSLDW | 0.65 | 50.7 | 0.99 |
| 6C2 | PD | — | GGDIKVSLDR | 0.73 | 48.2 | 0.797 |
| 6C3 | PD | — | GGDITLGLDV | 1.53 | 21.5 | 0.195 |
| 6G4 * | PD | — | GGDILDSLHF | 2.03 | 16.2 | 1.69 |
| CD20 | control | — | — | N/A | N/A | N/A |
| M4 | parental | — | — | 33 | N/A | 18.69 |
| 10H6 (a) | RbD | R18G(FW1); R24Q(CDR1) | F106L(CDR3); V117E(FW4) | 0.3 | 110 | 1.36 |
| 8G9 | RbD | — | G165S(FW1); F106L(CDR3) | 2.4 | 13.75 | 1.665 |
| 10D8 (b) | RbD | R18K(FW1); I31F(CDR1) | G10E(FW1) | 2.6 | 12.7 | 2.117 |
| 3B3 | RbD | I31F(CDR1) | F106L(CDR1) | 2.2 | 15 | 2.13 |
| 1G6 | RbD | I31F(CDR1) | T104N(CDR3) | 1.2 | 21.5 | 2.914 |
| 2E6 (c) | RbD | — | G9V(FW1);F106L; T104N(CDR3) | 1.9 | 17.3 | 2.708 |
| 5A3 | RbD | R181(FW1); G68S(FW3) | Q82K; A97T(FW3); F106L(CDR3) | 3 | 11 | 1.532 |
| 10G10 | RbD | K103R(FW4) | T35S(FW2); D56E(CDR2) | 16.3 | 2 | 3.807 |
| 5H3 | RbD | Y49D(FW2) | Q3L(FW1) | 8.2 | 4 | 3.05 |

| | Fold gain scFv-Fc | ScFv-Fc | Yield | SEC scFv-Fc | SEC scFv | BIAcore kd * | Fold change |
|---|---|---|---|---|---|---|---|
| Clone ID | (hRAGE) | Mu-IC50 | (μg/ml) | (% monomer) | (% monomer) | scFv-Fc | in kd |
| CD20 | N/A | N/A | 8 | 55 | 89 | N/A | N/A |
| M4 | N/A | 5.31 | 21.1 | 94.9 | 58.8 | 2.93E−02 | N/A |
| 3A6 | 9.9 | 1.44 | 7.5 | Nd | 68.7 | 8.67E−04 | 33.8 |
| 3B2 | 34.2 | 0.3 | 9.1 | 30.33 | 67.3 | 6.84E−04 | 42.8 |
| 3B4 | 25 | 0.47 | 15.3 | 94.27 | 100 | 4.20E−04 | 69.8 |
| 3D2 | 31.3 | 0.63 | 11.7 | 61.36 | 71.1 | 3.91E−03 | 7.4 |
| 3G5 | 62.7 | 0.78 | 14.1 | 31.67 | 70.8 | 4.50E−04 | 65.1 |
| 6B2 | 8.2 | 2.4 | 12.5 | 71.9 | 65.6 | 4.84E−04 | 61.0 |
| 6B6 | 7.6 | 1.54 | 8.9 | Nd | 71.4 | 1.41E−03 | 20.8 |
| 6C2 | 9.4 | 1.44 | 12.1 | Nd | 68.6 | 1.53E−03 | 19.2 |
| 6C3 | 38.6 | 1.02 | 6.2 | 55.7 | 62.6 | 4.88E−04 | 60.0 |
| 6g4 * | 11.1 | 0.131 | 9.2 | 82.43 | 55.4 | 3.88E−03 | 7.6 |
| CD20 | N/A | N/A | 8 | 55 | 89 | N/A | N/A |
| M4 | N/A | 10.61 | 21.1 | 94.9 | 58.8 | 2.91E−02 | N/A |
| 10H6 (a) | 13.7 | 8.873 | 7.7 | 81.32 | 82.9 | 1.16E−03 | 25.3 |
| 8G9 | 5.1 | 9.624 | 5.5 | 81.67 | 70.2 | 3.44E−03 | 3.9 |
| 10D8 (b) | 8.8 | 7.728 | 7.7 | 95.87 | 86.8 | 5.66E−04 | 51.8 |
| 3B3 | 8.8 | 10.02 | 9.1 | 76.98 | 77.7 | 6.49E−03 | 4.5 |
| 1G6 | 6.4 | 7.212 | 9 | 91.54 | 84.6 | 4.38E−04 | 66.9 |
| 2E6 (c) | 6.9 | 7.502 | 9.1 | 83.42 | 76.6 | 1.14E−03 | 25.7 |
| 5A3 | 12.2 | 6.387 | 8.3 | 78.72 | 58.3 | 4.94E−03 | 5.9 |
| 10G10 | 5 | 15.77 | 8.9 | 74.32 | 67.3 | 1.05E−01 | 0.28 |
| 5H3 | 6.1 | 12.18 | 9.4 | 91.76 | 100 | 8.26E−02 | 0.35 |

PD—Phage display, RbD—Ribosome display
6G4 wass assayed seperately and fold gain was calculated using M4 parental IC50 value from ribosome display derived clones
# refers to VH-CDR3 sequence
* BIAcore dissociation constant (kd; sec⁻¹) was determined by fitting 1:1 models to binding curves for single 10 nM scFv-Fc preparations binding to directly immobilized HU-RAGE-SA (~100 RU)
(a) Clone 10H6 carries D108N linker mutation;
(b) Clone 10D8 carries G114N linker mutation;
(c) Clone 2E6 carries S122N linker mutation

What is claimed:

1. An isolated antigen binding polypeptide that binds specifically to Receptor for Advanced Glycation End-product (RAGE) and comprises: one or more complementarity determining regions (CDRs) selected from the group consisting of SEQ ID NO's 42 to 45 and SEQ ID NOs 76 to 81.

2. The antigen binding polypeptide of claim 1 that further comprises:
at least one of a variable light chain domain (V_{L}) that is a variant of SEQ ID NO:1 and a variable heavy chain domain (V_{H}) that is a variant of SEQ ID NO:2, wherein the variant V_{L} comprises at least one invariant framework residue selected from the group consisting of Q6, P8 and S10 in Framework 1; P44 and I48 in Framework 2; R61, S65, E81, and C88 in Framework 3; and G99 in Framework 4 or the V_{H} variant comprises at least one invariant framework residue selected from the group consisting of S7 and A24 in Framework 1, and V37 and Q39 in Framework 2.

3. The antigen binding polypeptide of claim 1 wherein one of the CDR's comprises an amino acid sequence selected from the group consisting of SEQ ID NO.'s 46, 47 and 48.

4. The antigen binding polypeptide of claim 1 which further comprises at least one of a V_{L} and a V_{H}, wherein the V_{L} comprises SEQ ID NO:1 and the V_{H} comprises SEQ ID NO:2, with one or more framework substitutions selected from the group consisting of: R18K, R18G, R18I or R185; Y49D; G68S; K103R, K103E, K103N, K103Q, K103D, K103H, K103W and K103Y in SEQ ID NO:1 and Q3L, Q3H or Q3R, Q3K, Q3W or Q3Y; G9V or G9D; G10E; G165; T35S; Q82K; Q82R, Q82H, Q82W or Q82Y; A97T or A97S; and V117E, V117L, V117M in SEQ ID NO:2.

5. The antigen binding polypeptide of claim 1 that is a polypeptide selected from the group consisting of a chimeric antibody, a humanized antibody, a human antibody, a single chain antibody, a tetrameric antibody, a tetravalent antibody, a multispecific antibody, a domain-specific antibody, a domain-deleted antibody, a fusion protein, an ScFc fusion protein, an Fab fragment, an Fab′ fragment, an F(ab′)2 fragment, an Fv fragment, an ScFv fragment, an Fd fragment, a single domain antibody, a dAb fragment, a small modular immunopharmaceutical (SMIP) a nanobody, a CDR3 peptide, and a constrained FR3-CDR3-FR4 peptide.

7. The antigen binding polypeptide of claim 1, wherein the polypeptide binds to the V domain of human RAGE.

8. The antigen binding polypeptide of claim 1, wherein the polypeptide binds to RAGE and inhibits the binding of a RAGE binding partner (RAGE-BP) to the RAGE.

9. The antigen binding polypeptide of claim 1 that reduces or prevents binding of antibody XT-M4 to RAGE.

10. The antigen binding polypeptide of claim 1 that comprises a V_{L} amino acid sequence selected from the group consisting of: SEQ ID NO:'s. 69 through 74.

11. The antigen binding polypeptide of claim 1, that comprises a V_{H} amino acid sequence selected from the group consisting of: SEQ ID NO:'s. 50 through 68.

12. The antigen binding polypeptide of claim 11 that further comprises a V_{L} amino acid sequence selected from the group consisting of: SEQ ID NO:'s. 69 through 74.

13. The antigen binding polypeptide of claim 1 that comprises an scFv.

14. The antigen binding polypeptide of claim 13 that further comprises at least one mutation of an amino acid in the V_{L} or V_{H} that removes a glycosylation site.

15. The antigen binding polypeptide of claim 14 that comprises a flexible linker between the V_{H} and the V_{L} chain domains.

16. The antigen binding polypeptide of claim 15 comprising in sequential order from the amino to carboxy end the V_{L} domain, the flexible linker and the V_{H} domain.

17. The antigen binding polypeptide of claim 15 comprising in sequential order from the amino to carboxy end the V_{H} domain, the flexible linker and the V_{L} domain.

18. The antigen binding polypeptide of claim 17 wherein the flexible linker comprises the linker of SEQ ID NO. 41 having at least one substitution selected from the group consisting of: D1N, G7N 515N.

19. The antigen binding polypeptide of claim 16 or claim 17 wherein the flexible linker comprises the amino acid sequence of SEQ ID NO: 41.

20. An antigen binding polypeptide that specifically binds to mouse and human RAGE and comprises a V_{L} amino acid sequence that is at least 90% identical to SEQ ID NO: 1 and a V_{H} amino acid sequence that is at least 90% identical to SEQ ID NO:2.

21. The antigen binding polypeptide of claim 16 that comprises an amino acid sequence selected from the group consisting of: SEQ ID No's 3-11 and SEQ ID No's 21-30.

22. The antigen binding polypeptide of claim 21 that is encoded by a nucleic acid selected from the group consisting of SEQ ID NO's 12-20 and SEQ ID NO's 31-40.

23. The antigen binding polypeptide of claim 1 that comprises an antibody or a fragment of an antibody.

24. The antigen binding polypeptide of claim 23 that comprises a V_{H} amino acid sequence selected from the group consisting of: SEQ ID NO:'s 50 to 68.

25. The antigen binding polypeptide of claim 23 that comprises a V_{L} amino acid sequence selected from the group consisting of: SEQ ID NO:'s 69 to 74.

26. The antigen binding polypeptide of claim 25 that further comprises a V_{L} amino acid sequence selected from the group consisting of: SEQ ID NO:'s 69 to 74.

27. The antigen binding polypeptide of claim 26, wherein the antibody further comprises at least one mutation of an amino acid in the V_{L} or V_{H} that removes a glycosylation site.

28. A pharmaceutical composition comprising the antibody or antibody fragment of claim 27 and a pharmaceutically acceptable excipient.

29. An isolated nucleic acid comprising a nucleotide sequence encoding at least one anti-RAGE antibody variable region amino acid sequence selected from the group consisting of SEQ ID NO;'s 50 through 74.

30. An isolated nucleic acid that specifically hybridizes to a nucleic acid of claim 36 or a complement thereof under stringent hybridization conditions.

31. A method of treating a subject having a RAGE-related disease or disorder comprising administering to the subject a therapeutically effective amount of the antigen binding polypeptide of claim 1.

32. The method of claim 31 wherein the antigen binding polypeptide is an antibody or a fragment of an antibody.

33. The method of claim 32 wherein the antibody or antibody fragment comprises a V_{H} domain comprising an amino acid sequence of any of SEQ ID NO:'s 50 to 68.

34. The method of claim 32 wherein the antibody or antibody fragment comprises a V_{L} domain comprising an amino acid sequence of any of SEQ ID NO:'s 69 to 74.

35. The method of claim 33 wherein the antibody or antibody fragment further comprises a V_{L} domain comprising an amino acid sequence of any of SEQ ID NO:'s 69 to 74.

36. The method of claim 31, wherein the RAGE-related disease or disorder is selected from the group consisting of sepsis, septic shock, listeriosis, inflammatory diseases, cancers, arthritis, Crohn's disease, chronic acute inflammatory diseases, cardiovascular diseases, erectile dysfunction, diabetes, complications of diabetes, vasculitis, nephropathies, retinopathies, and neuropathies.

37. The method of claim 36, comprising administering the antigen-binding polypeptide in combination with one or more agents useful in the treatment of the RAGE-related disease or disorder that is to be treated.

38. The method of claim 36, wherein the agent is selected from the group consisting of: anti-inflammatory agents, antioxidants, beta-blockers, antiplatelet agents, ACE inhibitors, lipid-lowering agents, anti-angiogenic agents, and chemotherapeutics.

39. The method of claim 36 wherein the RAGE-related disease is selected from the group consisting of sepsis, shock and listeriosis.

40. The method of claim 36 wherein the antigen binding polypeptide specifically binds to soluble RAGE (sRAGE).

41. The method of claim 40, wherein the antigen binding polypeptide specifically binds to sRAGE selected from the group consisting of murine sRAGE and human sRAGE.

42. The method of claim 41 wherein the antigen binding polypeptide is an antibody or a fragment of an antibody.

43. The method of claim 42 wherein the antibody or antibody fragment comprises a V_{H} domain comprising an amino acid sequence of any of SEQ ID NO:'s 50 to 68.

44. The method of claim 42 wherein the antibody or antibody fragment comprises a V_{L} domain comprising an amino acid sequence of any of SEQ ID NO:'s 69 to 74.

45. The method of claim 43 wherein the antibody or antibody fragment further comprises a V_{L} domain comprising an amino acid sequence of any of SEQ ID NO:'s 69 to 74.

46. A method of inhibiting the binding of a RAGE binding partner (RAGE-BP) to RAGE in a mammalian subject comprising administering to the subject an inhibitory amount of the antigen binding polypeptide of claim 1.

47. The method of claim 46 wherein the antigen binding polypeptide specifically binds to soluble RAGE (sRAGE).

48. The method of claim 46 wherein the antigen binding polypeptide is an antibody or a fragment of an antibody.

49. The method of claim 48 wherein the antibody or antibody fragment comprises a V_{H} domain comprising an amino acid sequence of any of SEQ ID NO:'s 50 to 68.

50. The method of claim 48 wherein the antibody or antibody fragment comprises a V_{L} domain comprising an amino acid sequence of any of SEQ ID NO:'s 69 to 74.

51. The method of claim 49 wherein the antibody or antibody fragment further comprises a V_{L} domain comprising an amino acid sequence of any of SEQ ID NO:'s 69 to 74.
